(19) United States
(12) Patent Application Publication
(10) Pub. No.: US 2022/0059214 A1
(43) Pub. Date: Feb. 24, 2022

(54) METHOD FOR TRAINING NUTRITIONAL ITEM RECOMMENDATION SYSTEM AND METHOD FOR RECOMMENDING NUTRITIONAL ITEMS

(71) Applicant: RATEGIA OY, Helsinki (FI)

(72) Inventors: Alexander JUNG, Helsinki (FI); Zahra ROUHANI, Helsinki (FI); Katja RATAMÄKI, Helsinki (FI); Taru PILVI, Helsinki (FI)

(21) Appl. No.: 17/299,329

(22) PCT Filed: Dec. 2, 2019
(86) PCT No.: PCT/FI2019/050862

§ 371 Date: Jun. 3, 2021

(30) Foreign Application Priority Data
Dec. 3, 2018 (FI) 20186037

(51) Int. Cl. G16H 20/60; G16H 50/20; G16H 50/70; G16H 10/60; G06Q 30/06

(52) U.S. Cl. CPC G16H 20/60; G16H 50/20; G06Q 30/0621; G16H 10/60; G06Q 30/0631; G16H 50/70

(57) ABSTRACT

A method for training a nutritional item recommendation server system and to a method for recommending a nutritional item. In the method, the nutritional item recommendation server system, once trained, is arranged to execute a machine learning recommendation algorithm and arranged to generate, by the trained machine learning recommendation algorithm of the nutritional item recommendation server system, a nutritional item recommendation output as a response to a recommendation request from a user based on the personal information of the user and nutritional item information.

### FIELD OF THE INVENTION

The present invention relates to a method for training a nutritional item recommendation server system for recommending nutritional items for users and more particularly to a method according to preamble of claim 1. The present invention further relates to a method recommending a nutritional item for a first user and more particularly to a method according to preamble of claim 11.

### BACKGROUND OF THE INVENTION

Different kinds of methods and algorithms are known in the prior art for recommending items to users. These prior art item recommendation methods and algorithms are arranged to recommend items based on similarity of items or based on user-item interaction of previous purchases, ratings or views.

Prior art methods and systems for recommending nutritional items are based on the above mentioned prior art methods or based on a pre-determined diet plan. These recommendation systems for recommending food items are general systems suitable for recommending any items in addition to or instead of food items.

One of the problems associated with the prior art is that the recommendation methods recommend items only based on preferences determined by the users or based on user actions, or based on preferences defined by the recommendation system itself.

A person may have personal physiological characteristics or restrictions and health or medical situations affecting suitable choices of food items. At the moment, the person has to go through each food item separately to inspect if the food item is suitable or not. Alternatively, the person may rely on pre-determined classifications of food items, such as gluten-free or lactose-free. Accordingly, the person may find food items in chosen pre-determined classification and then the person go through each food item separately if the food item is suitable or not. However, the pre-determined classification of food items is unable to take into account health effects or benefits to an individual person, for example which of the gluten-free breads would have greater health benefits to the individual person. Further, suitability of the food items to the individual person may change over time due to for example health situations, aging and physical activity. For the individual person, this means considering ingredients and nutrition information of the food items, own health situations, medication, allergies and so on. Even with this information, the individual person is unable to make decisions to choose more suitable food item within a certain group of food items, for example in group of breads.

### BRIEF DESCRIPTION OF THE INVENTION

An object of the present invention is to provide a method for providing a nutritional item recommendation system for recommending nutritional items for users and a method for recommending a nutritional item for a first user so as to solve or at least alleviate the prior art disadvantages. The objects of the invention are achieved by a method for training a nutritional item recommendation system which is characterized by what is stated in claim 1. The objects of the invention are further achieved by a method for recommending nutritional items to a first user which is characterized by what is stated in claim 11.

The preferred embodiments of the invention are disclosed in the dependent claims.

In the context of this application term “nutritional item” means food items or products, such as fruits, vegetables, bread, meat, food products, such as breads, dairy products, or ready-made meals, or recipes consisting of two or more food items. The term “nutritional item” further means food supplements, food replacement products, nutrition supplements or the like. The nutritional item may therefore comprise liquid food items, solid food items, raw food items, ready-cooked or half-cooked food items, pills, such as vitamins, or other similar products having nutritional value when consumed.

In the context of this application term “nutritional item category” means a group of similar nutritional items, such as a group of breads comprising several different kinds of breads.

The invention is based on the idea of providing a method for training a nutritional item recommendation system for recommending nutritional items for users, once trained the nutritional item recommendation server system being arranged to execute a machine learning recommendation algorithm.

The method comprises a step a) of receiving, in the nutritional item recommendation server system, personal information of plurality of users. The personal information comprising physiological information of plurality of users. The physiological information may comprise age, gender, height, weight, allergies, genetic information, medical information or other physiological information of the user.

The method also comprises a step b) of receiving, in the nutritional item recommendation server system, nutritional item information of plurality of nutritional items. The nutritional item information comprising nutrition and ingredients information of plurality of nutritional items. The nutrition information may comprise for example energy content, fat content, carbohydrate content, protein content, salt content or other physical nutrition information of the nutritional item. The ingredients information may comprise information of substances or nutritional substances used for producing the nutritional item.

The method further comprises a step c) of providing, in the nutritional item recommendation server system, a machine learning recommendation algorithm. The machine learning recommendation algorithm may be trained to recommend nutritional items to users.

The method comprises a step d) of applying, in the nutritional item recommendation server system, the personal information of plurality of users and the nutritional item information of plurality of nutritional items as training data to the machine learning recommendation algorithm and generating, in the nutritional item recommendation server system, a trained machine learning recommendation algorithm.

Further the method comprises a step e) of generating, by the trained machine learning recommendation algorithm of the nutritional item recommendation server system, a nutritional item recommendation output as a response to a recommendation request from a user based on the personal information of the user and the nutritional item information. Accordingly, the recommendation server system trains the machine learning recommendation algorithm with the physiological information of plurality of users and with the physical nutrition and ingredients information of the nutritional items such that the machine learning recommendation algorithm, once trained, may provide physiologically suitable nutritional item recommendation output for a user upon recommendation request.

The method may further comprise a step f) of receiving, in the nutritional item recommendation server system, user feedback information from the user as a response to the nutritional item recommendation output, and a step g) of applying, in the nutritional item recommendation server system, the user feedback information as reinforcement training data to the machine learning recommendation algorithm. Therefore, the machine learning recommendation algorithm may be a reinforcement machine learning algorithm which is further trained by utilizing user feedback relating to the recommendation output. The recommendation output may be recommended nutritional item. The user feedback information may comprise user purchase information, user rating information, user clicking information or any other information relating user reactions relating to the recommendation output.

The method for training the nutritional item recommendation server system may also comprise a step h) of receiving, in the nutritional item recommendation server system, user-item interaction information, the user-item information comprising plurality of interactions associated between users and nutritional items, and a step i) of applying, in the nutritional item recommendation server system, the user-item interaction information as training data to the machine learning recommendation algorithm and generating, in the nutritional item recommendation server system, the trained machine learning recommendation algorithm. Accordingly, the machine learning recommendation algorithm may be trained by utilizing information of nutritional items in relation to users. Each user-item interaction being associated with a user and nutritional item. This user-item interaction information may comprise user shopping history, user ratings, clicks, searches or the like user actions associated with nutritional items.

In one embodiment, the method further comprises receiving, in the nutritional item recommendation server system, nutritional item selection information from plurality of users in step f), the nutritional item selection information comprising one or more selected nutritional item and nutrition and ingredients information of the one or more selected nutritional items, and applying, in the nutritional item recommendation server system, the nutritional item selection information of plurality of users as reinforcement training data to the machine learning recommendation algorithm in step g).

In an alternative embodiment, the method further comprises maintaining, in the nutritional item recommendation server system, nutritional item selection history of the plurality of users, and applying, in the nutritional item recommendation server system, the nutritional item selection history of plurality of users as training data to the machine learning recommendation algorithm in step d) or as reinforcement training data to the machine learning recommendation algorithm in step g).

In yet alternative embodiment, the method comprises maintaining, in the nutritional item recommendation server system, nutritional item selection history of plurality of users, receiving, in the nutritional item recommendation server system, nutritional item selection information from plurality of users in step f), the nutritional item selection information comprising one or more selected nutritional item and nutrition and ingredients information of the one or more selected nutritional item, providing, in the nutritional item recommendation server system, the nutritional item selection information from plurality of users to the nutritional item selection history of plurality of users, and applying, in the nutritional item recommendation server system, the nutritional item selection information of plurality of users as training data to the machine learning recommendation algorithm in step d) or as reinforcement training data to the machine learning recommendation algorithm in step g).

Utilizing the selection history as training data or as reinforcement data enables generating recommendations which take into account past nutrition intake of the users. Thus, the nutritional item recommendations are also based on the recent nutrition intake and the recommendation may guide the user such that necessary nutrients may be obtained.

In one embodiment, the step e) comprises generating the nutritional recommendation output as response to a recommendation request of the nutritional item in a nutritional item category. Thus, the recommendation request comprises a nutritional item category information and the nutritional item recommendation output comprises one or more recommended nutritional items in the nutritional item category.

In one embodiment, the step e) comprises generating, by the trained machine learning recommendation algorithm of the nutritional item recommendation server system, the nutritional item recommendation output as the response to the recommendation request of the nutritional item in a pre-determined nutritional item category from a user based on the personal information of the user and the nutritional item information, the recommendation request comprising a nutritional item category information of the pre-determined nutritional item category and the nutritional item recommendation output comprising one or more recommended nutritional items in the pre-determined nutritional item category.

In an alternative embodiment, the step e) comprises generating, by the trained machine learning recommendation algorithm of the nutritional item recommendation server system, a nutritional item category based on the recommendation request of the nutritional item and the personal information of the user. The step e) further comprises generating, by the trained machine learning recommendation algorithm of the nutritional item recommendation server system, the nutritional item recommendation output as the response to the recommendation request of the nutritional item in the nutritional item category from a user based on the personal information of the user and the nutritional item information, the nutritional item recommendation output comprising one or more recommended nutritional items in the generated nutritional item category.

The category information provides recommendations only in the requested category or in the category which is suitable for the user.

The step a) of the method may further comprise:
receiving, in the nutritional item recommendation server system, user electronic health records of the plurality of users, the electronic health records comprising one or more measured physiological values of plurality of users; and
applying, in the nutritional item recommendation server system, the user electronic health records as training data to the machine learning recommendation algorithm and generating, in the nutritional item recommendation server system, the trained machine learning recommendation algorithm.

The physiological values may be measured with a physiological measurement device such as an activity monitor, a heart rate monitor, a blood pressure measurement device, a blood sugar monitor or the like. Thus, the method may comprise measuring, with a physiological measurement device, one or more physiological values of plurality of users. Measurement data from the physiological measurement device may be used as electronic health records. It should be noted, that the physiological measurement device may be arranged to connect with the nutritional item recommendation server system via a communications network for transferring the measured physiological values or the measured physiological values may be separately transmitted via a communication network to the nutritional item recommendation server system, for example via user interface in a user device. Further, the electronic health records or the measured physiological values may be generated with laboratory tests or measurements, such as blood tests or the like, and transmitted via a communication network to the nutritional item recommendation server system, for example via user interface in a user device.

Electronic health records may thus comprise any physiological values which may be measured from the user including genetic information of the user.

Therefore, the measured physical value(s) associated with users may be used as training data in the machine learning recommendation algorithm and thus the nutritional item recommendation server system may learn to recommend nutritional items based on the measured physical values of the users. Each electronic health data received in the recommendation server system, may be associated with a user.

The method may further comprise a step j):
receiving, in the nutritional item recommendation server system, general medical information, the general medical information comprising general medical health records;
providing, in the nutritional item recommendation server system, a machine learning grouping algorithm; and
applying, in the nutritional item recommendation server system, the general medical information as training data to the machine learning grouping algorithm and providing, in the nutritional item recommendation server system a trained machine learning grouping algorithm trained with the general medical information.

The general medical information or the general medical health records may comprise medical records of a certain population or a certain group of people. The general medical information may for example comprise hospital medical information. This general medical information is preferably anonymous, but each medical record is associated with an anonymous person with age, gender or the like personal information. The general medical information further comprises illness or sickness history data, measured physiological values, diagnosis or the like medical technical data associated with an anonymous user for entire group of people. Alternatively or additionally, the general medical information may comprise reference ranges for physiological values relating to health of people. The reference ranges may be based on standard values used in the health industry or in healthcare.

When the reference ranges are used, machine learning grouping algorithm may comprise only the clustering algorithm and the user is grouped to healthiness group based on the reference ranges and the user personal information by utilizing a clustering algorithm, preferably trained with the reference ranges.

The trained machine learning grouping algorithm of the nutritional item recommendation server system is arranged to cluster or classify users in healthiness group based their personal information and/or electronic health records. This information may be further used in the machine learning recommendation algorithm for recommending more suitable nutritional items based on the healthiness of the user. Further, this classification or grouping information may be used for sifting persons from one healthiness group to another healthiness group with better health values by nutritional item recommendations.

The method may further comprise a step k):
receiving, in the nutritional item recommendation server system, personal information of the user;
applying, in the nutritional item recommendation server system, the personal information of the user as grouping input data to the trained machine learning grouping algorithm;
generating, by the trained machine learning grouping algorithm of the nutritional item recommendation server system, a user healthiness output of the user based on the personal information of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm.

Alternatively, the method may comprise a step k):
receiving, in the nutritional item recommendation server system, personal information of the user and the electronic health records of the user;
applying, in the nutritional item recommendation server system, the personal information of the user and the electronic health records of the user as grouping input data to the trained machine learning grouping algorithm;
generating, by the trained machine learning grouping algorithm of the nutritional item recommendation server system, a user healthiness output of the user based on the personal information of the user and the electronic health records of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm.

Further alternatively, the method may comprise a step k):
receiving, in the nutritional item recommendation server system, the electronic health records of the user;
applying, in the nutritional item recommendation server system, the electronic health records of the user as grouping input data to the trained machine learning grouping algorithm;
generating, by the trained machine learning grouping algorithm of the nutritional item recommendation server system, a user healthiness output of the user based on the electronic health records of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm.

According to the above mentioned, the machine learning grouping algorithm may be used by applying personal information and/or the electronic health records associated to the user or a user account of the user and then generating a healthiness output associated to the user. The healthiness output comprises the classification of the user to a certain healthiness group based on the user personal information and/or the electronic health records of the user. Thus, the classification to the certain healthiness group is carried out using physiological information of the user or the measured physiological values of the user.

Accordingly, the person information and/or electronic health records of plurality of users may be applied to the machine learning grouping algorithm and healthiness output may be generated for plurality of users. Then, the healthiness output of the plurality of users may be applied to the machine learning recommendation algorithm as training date for training the machine learning recommendation algorithm with healthiness information of plurality of users. Each healthiness output being associated to a user.

The method may further comprise step l):
maintaining, in the nutritional item recommendation server system, a pre-evaluated user healthiness output;
upon the recommendation request from the user, receiving, in the nutritional item recommendation server system, personal information of the user;
generating, by the trained machine learning grouping algorithm of the nutritional item recommendation server system, the user healthiness output of the user by applying the personal information of the user to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user; and
applying, in the nutritional item recommendation server system, the health feedback information as reinforcement training data to the machine learning recommendation algorithm.

Alternatively, the method may comprise a step l):
maintaining, in the nutritional item recommendation server system, a pre-evaluated user healthiness output;
upon the recommendation request from the user, receiving, in the nutritional item recommendation server system, personal information of the user and the user electronic health records;
generating, by the trained machine learning grouping algorithm of the nutritional item recommendation server system, the user healthiness output of the user by applying the personal information of the user and the user electronic health records to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user;
applying, in the nutritional item recommendation server system, the health feedback information as reinforcement training data to the machine learning recommendation algorithm.

Further alternatively, the method may comprise a step l):
maintaining, in the nutritional item recommendation server system, a pre-evaluated user healthiness output;
upon the recommendation request from the user, receiving, in the nutritional item recommendation server system, the user electronic health records;
generating, by the trained machine learning grouping algorithm of the nutritional item recommendation server system, the user healthiness output of the user by applying the user electronic health records to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user;
applying, in the nutritional item recommendation server system, the health feedback information as reinforcement training data to the machine learning recommendation algorithm.

According to the above mentioned, the trained machine learning grouping algorithm may be utilized for generating feedback and reinforcement training date to the machine learning recommendation algorithm. For generating the health feedback information pre-determined user healthiness output may be stored in association of a user account. The nutritional item recommendation server system may also comprise purchase history of the user associated stored in association of the user account. The purchase history may for example comprise purchases after generating and storing the pre-determined user healthiness output, or after time stamp generated by the nutritional item recommendation server system for the pre-determined user healthiness output. Then, this purchase history may be applied together with the health feedback information as training data to the machine learning recommendation algorithm. Accordingly, the machine learning recommendation algorithm may be trained to recommend nutritional items such that the physiological values of the user may be enhanced, and also such that the user may be shifted from one user healthiness group to another.

In the present invention, the machine learning recommendation algorithm may be a network based machine learning algorithm, or a model based machine learning algorithm, or an artificial neural network or a non-parametric machine learning algorithm.

The machine learning recommendation algorithm may be a machine learning algorithm implementing reinforcement learning, or network based machine learning recommendation algorithm implementing reinforcement learning.

Further, the machine learning recommendation algorithm may be a model based machine learning algorithm, or an artificial neural network, a non-parametric machine learning algorithm implementing reinforcement learning. The network based machine learning algorithms, or model based machine learning algorithms, or artificial neural networks or the non-parametric machine learning algorithms are advantageous as they are able to process large amounts of data in fairly short period of time. Reinforcement learning enables the recommendation algorithm to develop for better recommendations based on user feedback and especially based on changes in user health.

Further, in the present invention the machine learning grouping algorithm may be a network based machine learning algorithm, or a model based machine learning algorithm, or an artificial neural network, or a non-parametric machine learning algorithm.

Alternatively, the machine learning grouping algorithm may be a network based machine learning algorithm, or a model based machine learning algorithms, or an artificial neural network, or a non-parametric machine learning algorithm implementing supervised learning.

In one embodiment, the machine learning grouping algorithm may comprise a network based machine learning algorithm, or a model based machine learning algorithm, or an artificial neural network or a non-parametric machine learning algorithm, and a clustering algorithm. In a further alternative embodiment, the machine learning grouping algorithm may comprise a network based machine learning algorithm, or a model based machine learning algorithm, or an artificial neural network or a non-parametric machine learning algorithm implementing supervised learning, and a clustering algorithm. In these embodiment, the machine learning algorithm implementing supervised learning first provides or outputs first healthiness output comprising probabilities for certain sicknesses or diseases for the user. Based on the first healthiness output, the clustering algorithm outputs the healthiness output of the user and places the user to a healthiness group.

Supervised learning enables the machine learning grouping algorithm to be trained only once or only periodically. This way the user healthiness outputs may remain comparable between different users and over time.

It should be noted that the user feedback information and the user healthiness output may be combined for generating evaluation feedback information which may be applied to the in the nutritional item recommendation server system, the evaluation feedback information as reinforcement training data to the machine learning recommendation algorithm. Therefore, the method may comprise:
receiving, in the nutritional item recommendation server system, user feedback information from the user as the response to the nutritional item recommendation output;
receiving, in the nutritional item recommendation server system, the user healthiness output of the user from the health server system;
generating, by the nutritional item recommendation server system, healthiness feedback information by comparing the personal information of the user with the user healthiness output of the user; and
generating, by the nutritional item recommendation server system, evaluation feedback information by combining the user feedback information and the healthiness feedback information; and
applying, in the nutritional item recommendation server system, the evaluation feedback information as reinforcement training data to the machine learning recommendation algorithm.

The user healthiness output may also be the healthiness feedback information as such.

The objects of the present invention may further be achieved with a method for recommending a nutritional item for a first user, the method being executable by a nutritional item recommendation server system executing a machine learning recommendation algorithm, once trained. The machine learning recommendation algorithm is trained by providing:
personal information of plurality of users as training data to the machine learning recommendation algorithm of the nutritional item recommendation server system, the personal information comprising physiological information of plurality of users, and nutritional item information of plurality of nutritional items as training data to the machine learning recommendation algorithm of the nutritional item recommendation server system, the nutritional item information comprising nutrition and ingredients information of plurality of nutritional items.

The method for recommending nutritional items may comprise:
a step A) of receiving, in the nutritional item recommendation server system, personal information of a first user, the personal information comprising the personal physiological information;
a step B) of receiving, in the nutritional item recommendation server system, a nutritional item recommendation request of a nutritional item from a first user associated with a first user account;
a step C) of applying, in the nutritional item recommendation server system, the nutritional item recommendation request and the personal information of the first user as recommendation input data to the trained machine learning recommendation algorithm;
a step D) of generating, by the trained machine learning recommendation algorithm of the recommendation server system, a nutritional item recommendation output as a response to the recommendation request from the first user based on the recommendation request and the personal information of the first user, the nutritional item recommendation output comprising one or more nutritional items.

According to the present invention, the nutritional item recommendation output is generated based on the physiological information of the user and the nutrition and ingredients information of the nutritional items. Accordingly, the method is capable of recommending nutritional items having most suitable ingredients and nutrition content relating to the physiological information of the user.

In one embodiment, the step A) comprises maintaining, in the nutritional item recommendation server system, plurality of user accounts, each user account comprising personal information of a user associated with the user account, the personal information comprising the personal physiological information. Further, the step B) comprises receiving, in the nutritional item recommendation server system, the nutritional item recommendation request of the nutritional item from the first user associated with the first user account.

Accordingly, instead of receiving the information of the first user upon the recommendation request, the personal information may be stored to a user account in the nutritional item recommendation server system.

In one embodiment, the step D) comprises generating, by the trained machine learning recommendation algorithm of the nutritional item recommendation server system, the nutritional item recommendation output as the response to the recommendation request of the nutritional item in a pre-determined nutritional item category from the first user based on the personal information of the first user and the nutritional item information, the recommendation request comprising a nutritional item category information of the pre-determined nutritional item category and the nutritional item recommendation output comprising one or more recommended nutritional items in the pre-determined nutritional item category.

Alternatively, the step D) comprises generating, by the trained machine learning recommendation algorithm of the nutritional item recommendation server system, a nutritional item category based on the recommendation request of the nutritional item and the personal information of the first user. The step D) further comprises generating, by the trained machine learning recommendation algorithm of the nutritional item recommendation server system, the nutritional item recommendation output as the response to the recommendation request of the nutritional item in the nutritional item category from the first user based on the personal information of the user and the nutritional item information, the nutritional item recommendation output comprising one or more recommended nutritional items in the generated nutritional item category.

Accordingly, the nutritional category information may be provided based on the recommended request and be based on the nutritional item of the recommendation request. Alternatively, the nutritional item category may be generated based on the recommendation request and the personal information of the user.

The method may also comprise a step F) of receiving, in the recommendation server system, user feedback information from the first user as a response to the nutritional item recommendation output, and applying, in the recommendation server system, the user feedback information as a reinforcement training data to the machine learning recommendation algorithm.

Thus, the method may comprise training the machine learning recommendation algorithm based on the user actions, for example purchases or choices relating to the recommendation outputs.

The method for recommending nutritional items may further comprise:
a step G) of receiving, in the nutritional item recommendation server system, user electronic health records of the first user;
applying, in the nutritional item recommendation server system, the user electronic health record of the first user as recommendation input data to the trained machine learning recommendation algorithm in step C); and
generating, by the trained machine learning recommendation algorithm of the recommendation server system, the nutritional item recommendation output as the response to the recommendation request from the first user based on the recommendation request, the personal information of the first user and the electronic health record of the first user in step D).

Alternatively, the method may comprise:
a step H) of measuring, by a physiological measurement device, one or more physiological values of the first user;
a step G) of receiving, in the nutritional item recommendation server system, the one or more measured physiological values of the first user as user electronic health records of the first user;
applying, in the nutritional item recommendation server system, the user electronic health records of the first user as recommendation input data to the trained machine learning recommendation algorithm in step C); and
generating, by the trained machine learning recommendation algorithm of the recommendation server system, the nutritional item recommendation output as response to the recommendation request from the first user based on the recommendation request, the personal information of the first user and the electronic health record of the first user in step D).

According to the above mentioned, the first user may provide electronic health records to the nutritional item recommendation server system and the electronic health records may be used for generating the recommendation output. This means, that the recommendation system may utilize measured physiological values of the first user for generating the recommendation output such that the recommendation output takes into account the measured physical values or physiological values and physiological state of the first user.

The machine learning recommendation algorithm may be trained by providing user electronic health records of plurality of users as training data to the machine learning recommendation algorithm of the nutritional item recommendation server system, the electronic health records comprising one or more measured physiological values of the plurality of users. This enables recommending nutritional items for example based on the activity level of the user based on activity measurement or nutritional products having low salt concentration based on measured high blood pressure.

Further, in the method for recommending nutritional items, the machine learning recommendation algorithm may be trained by providing user-item interaction information as training data to the machine learning recommendation algorithm of the nutritional item recommendation server system, the user-item information comprising plurality of interactions associated between users and nutritional items.

Alternatively, the machine learning recommendation algorithm may be trained by providing user-item interaction information as training data to the machine learning recommendation algorithm of the nutritional item recommendation server system, the user-item information comprising plurality of interactions associated between users and nutritional items. The method may further comprise receiving, in the nutritional item recommendation server system, user feedback information from the first user as the response to the nutritional item recommendation output and applying, in the recommendation server system, the user feedback information as the reinforcement training data to the machine learning recommendation algorithm.

This allows the nutritional item recommendation system to learn from the actions of plurality of users and recommend nutritional items taking into account the previous actions of plurality of users. This may enable recommending nutritional items more suitable to users based on the user behavior and actions. For example, this may reduce recommendations of nutritional items which are rejected by users.

Nutritional item selection information or nutritional item selection history of the first user may be utilized as the user-item interaction information.

In the present invention, the method may comprise a step I) of evaluating healthiness of the first user upon recommendation request from first user, the evaluating healthiness of the first user being executable by the nutritional item recommendation server system executing a machine learning grouping algorithm, once trained. The machine learning grouping algorithm being trained by providing general medical information as training data to the machine learning grouping algorithm of the nutritional item recommendation server system, the general medical information comprising general medical health records.

The method may further comprise in step I):
receiving, in the nutritional item recommendation server system, personal information of the first user;
applying, in the nutritional item recommendation server system, the personal information of the first user as grouping input data to the trained machine learning grouping algorithm;
generating, by the trained machine learning grouping algorithm of the nutritional item recommendation server system, a user healthiness output of the first user based on the personal information of the first user.

Alternatively, the method may comprise in step I):
receiving, in the nutritional item recommendation server system, electronic health records of the first user;
applying, in the nutritional item recommendation server system, the electronic health records of the first user as grouping input data to the trained machine learning grouping algorithm;
generating, by the trained machine learning grouping algorithm of the nutritional item recommendation server system, a user healthiness output of the first user based on the electronic health records of the first user.

Yet alternatively the method may comprise in step I):
receiving, in the nutritional item recommendation server system, personal information of the first user and electronic health records of the first user;
applying, in the nutritional item recommendation server system, the personal information of the first user and the electronic health records of the first user as grouping input data to the trained machine learning grouping algorithm;
generating, by the trained machine learning grouping algorithm of the nutritional item recommendation server system, a user healthiness output of the first user based on the personal information of the first user and the electronic health records of the first user.

Accordingly, the first user may be clustered or classified to the user healthiness group based on the personal information and/or electronic health records of the first user. The grouping may then be done based on the physiological information and/or measured physiological values of the first user.

The method may also comprise a step J):
applying, in the nutritional item recommendation server system, the user healthiness output of the first user as recommendation input data to the trained machine learning recommendation algorithm; and
generating, by the trained machine learning recommendation algorithm of the nutritional item recommendation server system, the nutritional item recommendation output as the response to the recommendation request from the first user based on the recommendation request, the personal information of the first user and the user healthiness output of the first user, or based on the recommendation request, the personal information of the first user, the electronic health record of the first user and the user healthiness output of the first user.

Accordingly, the user healthiness output may be further utilized in the machine learning recommendation algorithm as input data such that the physical healthiness of the first user may be taken into account in the recommendation for recommending nutritional items with suitable nutrition and ingredients.

The method may also comprise a step J):
maintaining, in the nutritional item recommendation server system, a pre-evaluated user healthiness output of the first user;
comparing, in the nutritional item recommendation server system, the pre-evaluated user healthiness output of the first user and the user healthiness output of the first user;
generating, by the nutritional item recommendation server system, health feedback information based on the comparison of the pre-evaluated user healthiness output of the first user and the user healthiness output of the first user;
applying, in the nutritional item recommendation server system, the health feedback information as reinforcement training data to the machine learning recommendation algorithm.

This allows determining change in the healthiness of the first user and use that information for training the machine learning recommendation algorithm based on the change. This allows the recommendations to take into account changes in the physiological information and/or measured physiological values of the first user.

In the method, the machine learning recommendation algorithm may be a machine learning algorithm implementing reinforcement learning, or a network based machine learning recommendation algorithm implementing reinforcement learning. Alternatively, the machine learning recommendation algorithm may be an artificial neural network, non-parametric machine learning algorithm implementing reinforcement learning.

In the method, the machine learning grouping algorithm may be a machine learning algorithm implementing supervised learning algorithm, or a machine learning algorithm implementing supervised learning. Alternatively, the machine learning grouping algorithm may be a model based machine learning algorithm, or an artificial neural network or non-parametric machine learning algorithm implementing supervised learning. The machine learning grouping algorithm may also comprise machine learning algorithm, or a model based machine learning algorithm, or an artificial neural network or non-parametric machine learning algorithm implementing supervised learning, and a clustering algorithm clustering the results or outputs of the mentioned machine learning grouping algorithm implementing supervised learning.

In some embodiments, the machine learning recommendation algorithm is further trained by:
receiving, in the nutritional item recommendation server system, nutritional item selection information from the first user, the nutritional item selection information comprising one or more selected nutritional item and nutrition and ingredients information of the one or more selected nutritional item; and
applying, in the nutritional item recommendation server system, the nutritional item selection information of the first user as training data to the machine learning recommendation algorithm and generating.

Alternatively, the machine learning recommendation algorithm is further trained by:
maintaining, in the nutritional item recommendation server system, nutritional item selection history of the first user, the nutritional item selection history being associated to the first user account of the first user; and
applying, in the nutritional item recommendation server system, the nutritional item selection history of the first user as training data to the machine learning recommendation algorithm.

In yet alternative embodiments, the machine learning recommendation algorithm is further trained by:
maintaining, in the nutritional item recommendation server system, nutritional item selection history of the first user, the nutritional item selection history being associated to the first user account of the first user;
receiving, in the nutritional item recommendation server system, nutritional item selection information from the first user, the nutritional item selection information comprising one or more selected nutritional item and nutrition and ingredients information of the one or more selected nutritional item;
providing, in the nutritional item recommendation server system, the nutritional item selection information from the first user to the nutritional item selection history of the first user; and
applying, in the nutritional item recommendation server system, the nutritional item selection information of the first user as training data to the machine learning recommendation algorithm and generating.

Training the machine learning recommendation algorithm with nutritional item selection information of selection history of enables providing recommendations which take into account the nutrients and ingredients of nutritional item selections made by the users. Thus, necessary nutrients consumption and allocation may be enhanced by providing recommendations more suitable for current situation of the users upon receiving the recommendation request.

In some embodiments of the present invention, the method further comprises a step K):
maintaining, in the nutritional item recommendation server system, nutritional item selection history of the first user, the nutritional item selection history being associated to the first user account of the first user;
in step C) applying, in the nutritional item recommendation server system, the nutritional item recommendation request, the personal information of the first user and the nutritional item selection history as input data to the trained machine learning recommendation algorithm; and
in step D) generating, by the trained machine learning recommendation algorithm of the recommendation server system, a nutritional item recommendation output as a response to the recommendation request from the first user based on the recommendation request, the personal information of the first user and the nutritional item selection history, the nutritional item recommendation output comprising one or more nutritional items in the nutritional item group.

In an alternative embodiment, the step K) comprises:
receiving, in the nutritional item recommendation server system, nutritional item selection information from the first user, the nutritional item selection information comprising one or more selected nutritional items and nutrition and ingredients information of the one or more selected nutritional items;
associating, in the nutritional item recommendation server system, nutritional item selection information from the first user to the first user account; and
in step C) applying, in the nutritional item recommendation server system, the nutritional item recommendation request, the personal information of the first user and the nutritional item selection information as input data to the trained machine learning recommendation algorithm; and
in step D) generating, by the trained machine learning recommendation algorithm of the recommendation server system (50), a nutritional item recommendation output as a response to the recommendation request from the first user based on the recommendation request, the personal information of the first user and the nutritional item selection information, the nutritional item recommendation output comprising one or more nutritional items in the nutritional item group.

In a further alternatively embodiment, the step K) comprises:
maintaining, in the nutritional item recommendation server system, nutritional item selection history of the first user, the nutritional item selection history being associated to the first user account of the first user;
receiving, in the nutritional item recommendation server system, nutritional item selection information from the first user, the nutritional item selection information comprising one or more selected nutritional item and nutrition and ingredients information of the one or more selected nutritional item;
providing, in the nutritional item recommendation server system, the nutritional item selection information from the first user to the nutritional item selection history of the first user;
in step C) applying, in the nutritional item recommendation server system, the nutritional item recommendation request, the personal information of the first user and the nutritional item selection history as input data to the trained machine learning recommendation algorithm; and
in step D) generating, by the trained machine learning recommendation algorithm of the recommendation server system, a nutritional item recommendation output as a response to the recommendation request from the first user based on the recommendation request, the personal information of the first user and the nutritional item selection history, the nutritional item recommendation output comprising one or more nutritional items in the nutritional item group.

Accordingly, past nutritional item selections of the first user are taken into account when providing a recommendation of the nutritional item. This is very specific in this context of health and nutritional items as the actual composition, ingredient and nutritional values of the food item which is to be recommended has an effect on the recommendation itself.

The present invention provides method for recommending nutritional items based on the user physiological information and the nutrition information and ingredient of the nutritional items. Accordingly, the present invention considers physiological state or characteristics of a user and provides recommendations based on the measurable characteristics of the users and nutritional items. Therefore, accurate and only suitable nutritional item recommendations maybe provided.

### BRIEF DESCRIPTION OF THE DRAWINGS

The invention is described in detail by means of specific embodiments with reference to the enclosed drawings, in which

FIG. 1 is a schematic view of one embodiment of a system according to the present invention;

FIG. 2 is a schematic view of another embodiment of a system according to the present invention;

FIG. 3 shows schematically a flow chart representing one embodiment of the present invention;

FIG. 4 shows schematically a flow chart representing another embodiment of the present invention;

FIG. 5 shows schematically a flow chart representing yet another embodiment of the present invention;

FIGS. 6 to 8 show schematically method steps according different embodiments of the present invention.

### DETAILED DESCRIPTION OF THE INVENTION

The invention and its embodiments are not specific to the particular information technology systems, communications systems and access networks, but it will be appreciated that the present invention and its embodiments have application in many system types and may, for example, be applied in a circuit switched domain, e.g., in GSM (Global System for Mobile Communications) digital cellular communication system, in a packet switched domain, e.g. in the UMTS (Universal Mobile Telecommunications System) system, and e.g. in networks according to the IEEE 802.11 standards: WLAN (Wireless Local Area networks), HomeRF (Radio Frequency) or BRAN (Broadband Radio Access Networks) specifications (HIPERLAN1 and 2, HIPERACCESS). The invention and its embodiments can also be applied in ad hoc communications systems, such as an IrDA (Infrared Data Association) network or a Bluetooth network. In other words, the basic principles of the invention can be employed in combination with, between and/or within any mobile communications systems of 2nd, 2,5th, 3rd, 4th and 5th (and be-yond) generation, such as GSM, GPRS (General Packet Radio Service), TETRA (Terrestrial Trunked Radio), UMTS systems, HSPA (High Speed Packet Access) systems e.g. in WCDMA (Wideband Code Division Multiple Access) technology, and PLMN (Public Land Mobile Network) systems.

Communications technology using IP (Internet Protocol) protocol can be, e.g., the GAN technology (General Access Network), UMA (Unlicensed Mobile Access) technology, the VoIP (Voice over Internet Protocol) technology, peer-to-peer networks technology, ad hoc networks technology and other IP protocol technology. Different IP protocol versions or combinations thereof can be used.

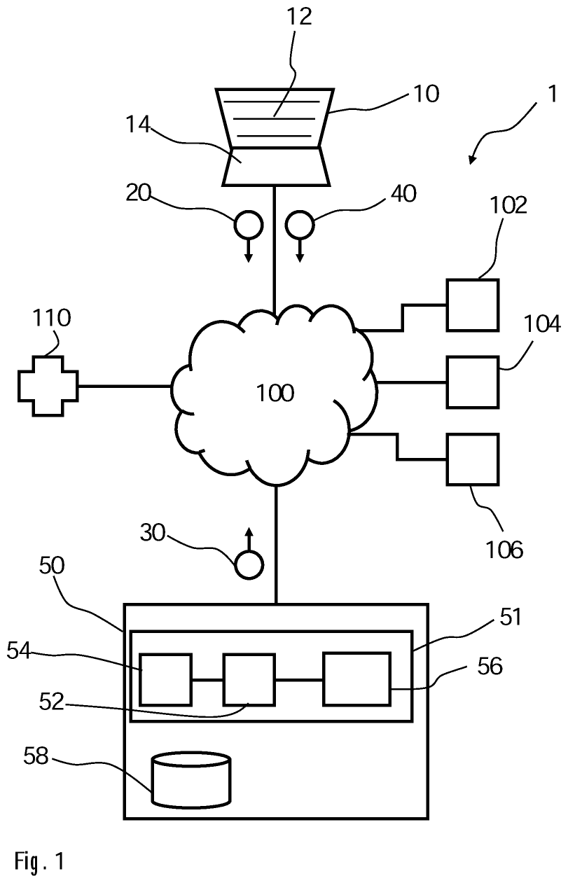

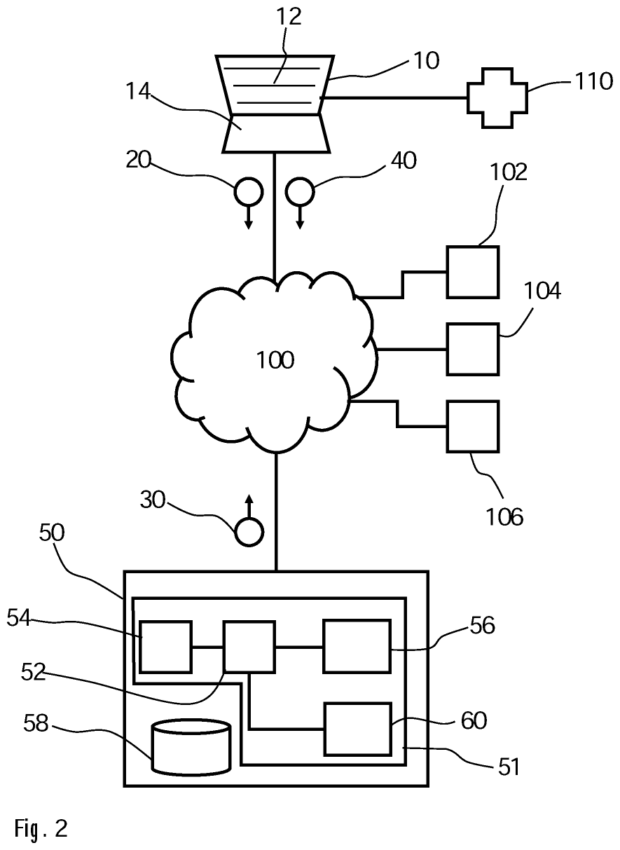

An architecture of a system to which embodiments of the invention may be applied is illustrated in FIGS. 1 and 2. FIGS. 1 and 2 illustrate simplified system architectures only showing some elements and functional entities, all being logical units whose implementation may differ from what is shown. The connections shown in the figures are logical connections; the actual physical connections may be different. It is apparent to a person skilled in the art that the systems also comprise other functions and structures.

According to the above mentioned, the present invention is not limited to any known or future systems or device or service, but may be utilized in any systems by following method according to the present invention.

Referring to FIG. 1, there is shown a schematic representation of a system 1, the system 1 being suitable for implementing non-limiting embodiments of the present invention. It is to be expressly understood that the system 1 as depicted is merely an illustrative implementation. Thus, the description thereof that follows is intended to be only a description of illustrative examples of the present technology. This description is not intended to define the scope or set forth the bounds of the present technology.

The system 1 is configured to provide nutritional item recommendations to a user of the system 1. The user may be a subscriber to a recommendation service provided by the system 1 and the user may have a user account provided in the system 1. However, the subscription does not need to be explicit or paid for. For example, the user can become a subscriber by virtue of downloading a nutritional item recommendation application from the system 1, by registering and provisioning a log-in/password combination, by registering and provisioning user preferences and the like. As such, any system variation configured to generate nutritional item recommendations for the given user can be adapted to execute embodiments of the present invention. Furthermore, the system 1 will be described using an example of the system 1 being a nutritional item recommendation system. However, embodiments of the present invention can be equally applied to other types of the systems 1, as will be described in greater detail herein below.

The system 1 comprises an electronic user device 10, the electronic user device 10 being associated with the user. As such, the electronic user device 10 can sometimes be referred to as a “client device”. It should be noted that the fact that the electronic user device 10 is associated with the user does not need to suggest or imply any mode of operation—such as a need to log in, a need to be registered, or the like.

The implementation of the electronic user device 10 is not particularly limited, but as an example, the electronic user device 10 may be implemented as a personal computer (desktops, laptops, netbooks, etc.), a wireless communication device (such as a smartphone, a cell phone, a tablet and the like), as well as network equipment (such as routers, switches, and gateways). The electronic user device 10 comprises hardware and/or software and/or firmware (or a combination thereof), as is known in the art, to execute a nutritional item recommendation application 14. The purpose of the nutritional item recommendation application 14 is to enable the user to receive (or otherwise access) nutritional item recommendations provided by the system 1, as will be described in greater detail herein below, and also send nutritional item recommendation requests to the system 1, as well as other possible information relating to the user.

Implementation of the nutritional item recommendation application 14 is not particularly limited. One example of the nutritional item recommendation application 14 may include a user accessing a web site associated with a nutritional item recommendation service to access the recommendation application 14. For example, the nutritional item recommendation application 14 can be accessed by typing in (or otherwise copy-pasting or selecting a link) an URL associated with the recommendation service. Alternatively, the nutritional item recommendation application 14 can be an app downloaded from a so-called app store, such as APPSTORE™ or GOOGLEPLAY™ and installed/executed on the electronic user device 10. It should be expressly understood that the nutritional item recommendation application 14 can be accessed using any other suitable means. In yet additional embodiments, the nutritional item recommendation application 14 functionality can be incorporated into another application, such as a browser application (not depicted) or the like. For example, the nutritional item recommendation application 14 can be executed as part of the browser application, for example, when the user first start the browser application, the functionality of the nutritional item recommendation application 14 can be executed.

The nutritional item recommendation application 14 may comprise a recommendation interface 12, the recommendation interface 12 being displayed on a screen of the electronic user device 10. In some embodiments of the present technology the recommendation interface 12 is presented when the user of the electronic user device 10 actuates (i.e. executes, run, background-run or the like) the nutritional item recommendation application 14. Alternatively, the recommendation interface 12 may be presented when the user opens a new browser window and/or activates a new tab in the browser application.

The recommendation interface 12 may comprise a search interface or search field. The search interface may comprise a search query interface for inputting nutritional item recommendation request, meaning nutritional item name such as bread.

The recommendation interface 12 may further comprise a nutritional item recommended content interface or field. The nutritional item recommended content field may comprise or display one or more recommended nutritional items, such as a first recommended nutritional item and a second recommended nutritional item.

The electronic user device 10 may be communicatively coupled to a communications network 100 for accessing a nutritional item recommendation server system 50. The communications network 100 may comprise one or more wireless networks, wherein a wireless network may be based on any mobile system, such as GSM, GPRS, LTE, 4G, 5G and beyond, and a wireless local area network, such as Wi-Fi. Furthermore, the communications network 100 may comprise one or more fixed networks or the Internet, or for short Bluetooth® and the like

The nutritional item recommendation server system 50 may be implemented as a conventional computer server. The nutritional item recommendation server system 50 may comprise at least one identification server 51 connected to a recommendation database 58. The nutritional item recommendation server system 50 may also comprise one or more other network devices (not shown), such as a terminal device, a server and/or a database. The nutritional item recommendation server system 50 may be configured to communicate with the one or more electronic user devices 10 via the communications network 100. The nutritional item recommendation server system 50 and the database 58 may form a single database server, that is, a combination of a data storage (database) and a data management system or they may be separate entities. The data storage may be any kind of conventional or future data repository, including distributed and/or centralized storing of data, a cloud-based storage in a cloud environment (i.e., a computing cloud), managed by any suitable data management system. The implementation of the data storage is irrelevant to the invention, and therefore not described in detail. In addition to or instead of the database 58, other parts of the nutritional item recommendation server system 50 may also be implemented as distributed server system comprising two or more separate servers or as a computing cloud comprising one or more cloud servers. In some embodiments, the nutritional item recommendation server system 50 may be a fully cloud-based server system. Further, it should be appreciated that the location of the nutritional item recommendation server system 50 is irrelevant to the invention. The nutritional item recommendation server system 50 may be operated and maintained using one or more other network devices in the system or using a terminal device (not shown) via the communications network 100.

The nutritional item recommendation server system 50 may also comprise a processing module 52. The processing module 52 is coupled to or otherwise has access to a memory module 54. The processing module 52 and the memory module 54 may form a recommendation server, or at least part of it. The recommendation server or the nutritional item recommendation server system 50, or the processing module 52 and/or the memory module 54, has access to the database 58. The processing module 52 may be configured to carry out instructions of a nutritional item recommendation sever application by utilizing instructions of the nutritional item recommendation server application. The nutritional item recommendation server application may be stored in the memory module 54 of the nutritional item recommendation server system 50. The nutritional item recommendation server application may comprise the instructions of operating the nutritional item recommendation server application. Thus, the processing module 52 may be configure to carry out the instructions of the nutritional item recommendation server application.

The processing module 52 may comprise one or more processing units or central processing units (CPU) or the like computing units. The present invention is not restricted to any kind of processing unit or any number of processing units. The memory module 54 may comprise non-transitory computer-readable storage medium or a computer-readable storage device. In some embodiments, the memory module 54 may comprise a temporary memory, meaning that a primary purpose of memory module 54 may not be long-term storage. Memory module 54 may also refer to a volatile memory, meaning that memory module 54 does not maintain stored contents when memory module 54 is not receiving power. Examples of volatile memories include random access memories (RAM), dynamic random access memories (DRAM), static random access memories (SRAM), and other forms of volatile memories known in the art. In some examples, memory module 54 is used to store program instructions for execution by the processing module 52, for example the nutritional item recommendation server application. Memory module 54, in one embodiment, may be used by software (e.g., an operating system) or applications, such as a software, firmware, or middleware. The memory module 54 may comprise for example operating system or software application, the message application, comprising at least part of the instructions for executing the method of the present invention.

It should be noted, that the database 58 may also configured to comprise software application, the nutritional item recommendation server application, comprising at least part of the instructions for executing the method of the present invention.

The database 58 may maintain information of user accounts of a plurality of users and/or user information/recommendation/user actions/shopping history uploaded or stored to the nutritional item recommendation server system 50 via said user accounts or user devices 10 or user interfaces 12 or user applications 14. The database 58 may comprise one or more storage devices. The storage devices may also include one or more transitory or non-transitory computer-readable storage media and/or computer-readable storage devices. In some embodiments, storage devices may be configured to store greater amounts of information than memory module 54. Storage devices may further be configured for long-term storage of information. In some examples, the storage devices comprise non-volatile storage elements. Examples of such non-volatile storage elements include magnetic hard discs, optical discs, solid-state discs, flash memories, forms of electrically programmable memories (EPROMs) or electrically erasable and programmable memories (EEPROMs), and other forms of non-volatile memories known in the art. In one embodiment, the storage device may comprise databases and the memory module 54 comprises instructions and operating message application for executing the method according to the present invention utilizing the processing module 52. However, it should be noted that the storage devices may also be omitted and the nutritional item recommendation server system 50 may comprise only the memory module 54. Alternatively, the memory module 54 could be omitted and the nutritional item recommendation server system 50 could comprise only one or more storage devices. Therefore, the terms memory module 54 and database 58 could be interchangeable in embodiments which they both are not present. The database 58 is operable with other components and data of the nutritional item recommendation server system 50 by utilizing instructions stored in the memory module 54 and executed by the processing module 52 over the communications network 100.

The database 58 may be provided in connection with the nutritional item recommendation server 51 or the nutritional item recommendation server 51 may comprise the database 58, as shown in FIG. 1. Alternatively, the database 58 may be provided as external database 58, external to the nutritional item recommendation server 51, and the database 58 may be connected to the nutritional item recommendation server 51 directly or via the communications network 100, and thus database 58 is provided to the nutritional item recommendation server system 50.

The storage device(s) may store one or more databases 58 for maintaining user account and information and data relating to users and user accounts. These different information items may be stored to different database blocks in the database 58, or alternatively they may group differently, for example based on each individual user account. The storage device(s) may store one or more databases 58 for maintaining nutritional items and information and data relating to nutritional items to be recommended or available for recommendation. These different information items may be stored to different database blocks in the database 58, or alternatively they may group differently, for example based on each nutritional item.

The nutritional item recommendation server system 50 or the nutritional item recommendation server 51 comprises a recommendation module 56. The recommendation module 56 has access to a data storage device 58 and to the memory module 54. The recommendation module 56 being configured to generate nutritional item recommendation as a response to recommendation request from a user via the electronic user device 10. The recommendation module 56 comprises a machine learning recommendation algorithm executable by the processing module 52. The recommendation module 56 may be provided in connection with the nutritional item recommendation server 51 or the nutritional item recommendation server 51 may comprise the recommendation module 56, as shown in FIG. 1. Alternatively, the recommendation module 56 may be provided as recommendation module 56, external to the nutritional item recommendation server 51, and the recommendation module 56 may be connected to the nutritional item recommendation server 51 directly or via the communications network 100, and thus the recommendation module 56 is provided to the nutritional item recommendation server system 50. The recommendation module 56 may comprise the machine learning recommendation algorithm which may be a network based machine learning algorithm, or a machine learning algorithm implementing reinforcement learning, or a network based machine learning recommendation algorithm implementing reinforcement learning. In one embodiment, the machine learning recommendation algorithm may be a network based, a model based machine learning algorithm, or non-parametric machine learning algorithm, or preferably an artificial neural network.

The machine learning recommendation algorithm, once trained, is trained to receive one or more recommendation input information items or recommendation input data and generate one or more nutritional item recommendation outputs based on the recommendation input data.

Also coupled to the communications network 100 may be multiple network resources, including a first network resource 102, a second network resource 104 and one or more additional network resources 106. The first network resource 102, the second network resource 104 and the one or more of additional network resources 106 are all network resources accessible by the electronic user device 10 via the communications network 100 and/or by the nutritional item recommendation server system 50. Respective content of first network resource 102, the second network resource 104 and the one or more of additional network resources 106 is not particularly limited.

At least one of the first network resource 102, the second network resource 104 and the one or more of additional network resources 106 can contain (or in other words, host) digital nutritional items. In some embodiments of the present invention, the content of the digital nutritional items may comprise but is not limited to: audio content for streaming or downloading, video content for streaming or downloading, picture content, text content or other multi-media content, or the like. Further, each digital nutritional item comprises or is associated with nutritional item information comprising nutrition and ingredients information of the nutritional item. The nutrition and ingredients information of the nutritional item represents the technical data of the physical nutritional item relating to the digital nutritional item. Accordingly, there may be more than one on-line shop or database connected to the nutritional item recommendation server system 50. Thus, the network resource 102, 104, 106 may be an on-line shop(s) or database(s). However, it should be noted, that the network resource may also be provided to or in connection with the nutritional item recommendation server 51 or server system 50 and the digital nutritional items may be stored to the database 58.

The content of the one or more network resources 102, 104, 106 may be “discoverable” to the electronic user device 10 by various means. For example, the user of the electronic user device 10 can use a browser application (not depicted) and enter a Universal Resource Locator (URL) associated with the given one of the first network resource 102, the second network resource 104 and the one or more of additional network resources 106. Alternatively, the user of the electronic user device 10 can execute a search using a search engine (not depicted) to discover the content of one or more of the first network resource 102, the second network resource 104 and the one or more of additional network resources 106. The search may be carried out by utilizing the user interface 12 of the electronic user device 10 and the nutritional item recommendation server system 50.

The nutritional item recommendation application 14 can recommend digital nutritional items available from the given one of the first network resource 102, the second network resource 104 and the one or more of additional network resources 106 to the user. The nutritional item recommendation server system 50 is configured to select digital nutritional items for the one or more recommendation digital nutritional items to be presented to the user via the nutritional item recommendation application 14. More specifically, the processing module 52 is configured to receive from the electronic user device 10 a recommendation request 20 and as a response to the recommendation request, generate a recommendation output 30 specifically customized for the user associated with the electronic user device 10 or the user account of the user. The processing module 52 can further coordinate execution of various routines described herein as performed by the recommendation module 56.

The recommendation request comprises a recommendation request for a nutritional item in a nutritional item category. The recommendation output comprises one or more recommended nutritional items in the nutritional item category in question.

In some embodiments of the present invention, the recommendation request 20 can be generated in response to the user providing an explicit indication of the user desire to receive the recommendation output 30. For example, the user interface 12 can provide a button (or another actuatable element) to enable the user to indicate a desire to receive a new or an updated recommendation output 30. As a non-limiting example, the user interface 12 can provide an actuable button that reads “Request recommendation” or “Search”. Within these embodiments, the recommendation request may be thought of as “an explicit request” in a sense of the user expressly providing the recommendation request. In this case, the user may input, and the nutritional item recommendation application 14 and further the nutritional item recommendation server system 50, may receive search term or search indication for a digital nutritional item.

In other embodiments, the recommendation request 20 may be generated in response to the user providing an implicit indication of the user desire to receive the recommendation output. In some embodiments of the present invention, the recommendation request 20 may be generated in response to the user starting the nutritional item recommendation application 14.

Alternatively, in those embodiments of the present technology where the nutritional item recommendation application 14 is implemented as a browser or a browser application, the recommendation request 20 may be generated in response to the user opening the browser application and may be generated, for example, without the user executing any additional actions other than activating the browser application. As another example, the recommendation request 20 may be generated in response to the user opening a new tab of the already-opened browser application and can be generated, for example, without the user executing any additional actions other than activating the new browser tab. In other words, the recommendation request 20 can be generated even without the user knowing that the user may be interested in obtaining a recommendation output 30.

The recommendation request 20 may also be generated in response to the user selecting a particular element of the browser application and may be generated, for example, without the user executing any additional actions other than selecting/activating the particular element of the browser application. Examples of the particular element of the browser application can include but are not limited to: an address line of the browser application bar; a search bar of the browser application and/or a search bar of a search engine web site accessed in the browser application; favorites or recently visited network resources pane; any other pre-determined area of the browser application interface or a network resource displayed in the browser application.

The recommendation module 56 or the nutritional item recommendation server system 50 may be configured to execute a “crawler” operation. In other words, the recommendation module 56 or the nutritional item recommendation server system 50 can execute a robot or instructions that “visits” a plurality of one or more network resources 102, 104, 106 and catalogues of one or more digital nutritional items hosted by a respective one of the network resources 102, 104, 106. In some embodiments of the present invention, the recommendation module 56 or the nutritional item recommendation server system 50 can catalogue the digital nutritional items into an index mapping a given digital item to a list of key words associated with the given digital item.

In alternative embodiments of the present invention, nutritional item recommendation server system 50 can share the functionality with another server (not depicted) and/or another service (not depicted). For example, the functionality nutritional item recommendation server system 50 can be shared with a search engine server (not depicted) executing a search engine service. When the nutritional item recommendation server system 50 crawls and indexes new resources that may potentially host digital nutritional items, the nutritional item recommendation server system 50 can also index such newly discovered (or updated) digital nutritional items for the purposes of the nutritional item recommendation server system 50 routines described herein.

The recommendation module 56 may be configured to execute one or more machine learning recommendation algorithms. In some embodiments of the present invention, one or more machine learning algorithms can be any suitable machine learning algorithm or reinforcement machine learning algorithm, such as but not limited to: a network based algorithm, or a model based algorithm, or an artificial neural network, or a network-based algorithm or a non-parametric algorithm.

The recommendation module 56 executes one or more machine learning recommendation algorithms to analyze the indexed digital nutritional items (i.e. those discovered and indexed by the nutritional item recommendation server system 50 or the recommendation module 56) to select one or more digital nutritional items as recommendation output for the user.

As shown in FIG. 1, one or more physiological measurement devices 110 may be connected to the nutritional item recommendation server system 50 via the communications network 100. The physiological measurement devices 110 may comprise heart rate monitors, activity monitors (including mobile phones) blood pressure monitors, laboratory equipment or the like physiological measurement devices capable of measuring physiological values of users. The physiological measurement devices are configured to measure the physiological values and generate electronic health records representing the measured values. The one or more physiological measurement devices 110 connectable directly to the communications network 110 such that the nutritional item recommendation server system 50 may receive the electronic health records from the one or more physiological measurement devices 110. Alternatively, the measured physiological values or the electronic health records may be provided by one of the network resources 102, 104, 106 such that the nutritional item recommendation server system 50 may receive the measured physiological values or the electronic health records from one or more of the external network resources 102, 104, 106. The one or more physiological measurement devices 110 be configured to upload the electronic health records to one or more of the external network resources 102, 104, 106, automatically or as a response to request from a user.

Further alternatively and as shown in FIG. 2, the one or more physiological measurement devices 110 may be connectable to the electronic user device 10. Thus, the electronic health records may be received from the one or more physiological measurement devices 110 to the electronic user device 10. Further, the nutritional item recommendation server system 50 may receive the electronic health records from the electronic user device 10, for example via the nutritional item recommendation application 14.

It should be noted that, the electronic health records may be automatically generated by the physiological measurement devices 110. Alternatively, the electronic health records may be generated based on the measured values with the one or more physiological measurement devices 110. In the latter case, the electronic health records may be generated with a separate software application automatically or by input by a user or some other person.

Accordingly, the electronic health records comprise measured physiological values or data of a user. However, the method of the present invention is not limited to any particular method or apparatus or system for generating the electronic health records.

Each electronic health record received in the nutritional item recommendation server system 50 is associated with a user or user account, or the user personal information.

FIG. 2 shows another embodiment of a system for carrying out the method(s) of the present invention. In this embodiment, the nutritional item recommendation server system 50 or the nutritional item recommendation server 51 comprises a healthiness module 60. The healthiness module 60 has access to the data storage device 58 and to the memory module 54, and the recommendation module 56. The healthiness module 60 is configured to generate healthiness evaluation of a user upon a recommendation request from the user via the electronic user device 10. Alternatively, healthiness module 60 may be configured to generate a pre-determined healthiness evaluation of a user based on user personal information and/or electronic health records of the user and store the pre-determined healthiness evaluation to the nutritional item recommendation server system 50 or the data storage device 58 and associated the pre-determined healthiness evaluation with the user or with the user account of the user.

The healthiness module 60 comprises a machine learning grouping algorithm executable by the processing module 52. The healthiness module 60 may be provided in connection with the nutritional item recommendation server 51 or the nutritional item recommendation server 51 may comprise the healthiness module 60, as shown in FIG. 2. Alternatively, the healthiness module 60 may be provided as healthiness module 60, external to the nutritional item recommendation server 51, and the healthiness module 60 may be connected to the nutritional item recommendation server 51 directly or via the communications network 100, and thus the healthiness module 60 is provided to the nutritional item recommendation server system 50. The healthiness module 60 may comprise the machine learning grouping algorithm which may be a network based machine learning algorithm, or a machine learning algorithm implementing supervised learning, or a network based machine learning grouping algorithm implementing supervised learning. In one embodiment, the machine learning grouping algorithm may be network-based, or model based machine learning algorithm, or a non-parametric machine learning algorithm, or preferably an artificial neural network.

The machine learning grouping algorithm, once trained, is trained to receive user personal information data and/or electronic health records of the user as input data and generate a healthiness output based on the user personal information data and/or electronic health records of the user.

The healthiness module 60 may be configured to execute one or more machine learning grouping algorithms. In some embodiments of the present invention, one or more machine learning algorithms can be any suitable machine learning algorithm or supervised machine learning algorithm, such as but not limited to: a network based algorithm, or a model based algorithm, or an artificial neural network, or a non-parametric algorithm.

The machine learning grouping algorithm of the healthiness module 60 is trained with general medical information. The general medical information comprising general medical health records or general reference ranges of physiological values. The physiological values being measurable directly or being generated via analysis of measured values.

The healthiness module 60 executes one or more machine learning grouping algorithms to evaluate user healthiness and to generate a user healthiness output of the user based on the personal information and/or the electronic health records of the user for grouping or classifying the user to a user healthiness group representing the healthiness of the user. The user personal information and/or the electronic health records of the user may be used as input data to the one or more machine learning grouping algorithms of the healthiness module 60.

The healthiness module 60 or the nutritional item recommendation server system 50 may be configured to execute a “crawler” operation. In other words, the healthiness module 60 or the nutritional item recommendation server system 50 can execute a robot or instructions that “visits” a plurality of one or more network resources 102, 104, 106 and catalogues of general medical information or general reference ranges hosted by a respective one of the network resources 102, 104, 106 for receiving training data, periodically or upon request. Alternatively, the machine learning grouping algorithm of the healthiness module 60 may be trained once.

The present invention relates to a method for training a nutritional item recommendation system 1, as shown in FIGS. 1 and 2, for recommending nutritional items for users. Once trained, the nutritional item recommendation system being arranged to execute a machine learning recommendation algorithm for recommending nutritional items to users. The present invention further relates to a method for recommending nutritional items to users.

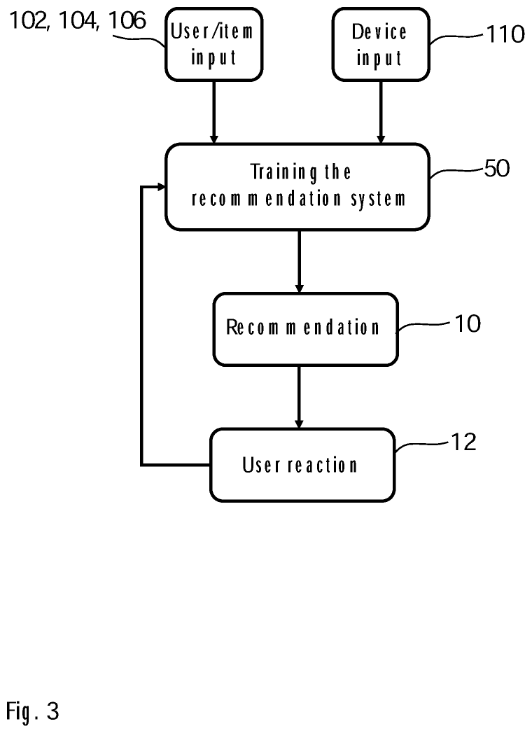

FIG. 3 shows schematically a flow chart according to one embodiment of the present invention for training the nutritional item recommendation system 1 such the once trained the nutritional item recommendation system is arranged to execute a machine learning recommendation algorithm.

The method comprises receiving, in the nutritional item recommendation server system 50, personal information of plurality of users as user input, the personal information comprising physiological information of plurality of users. The user input may be received from the network resources 102, 104, 106. Alternatively, the user input may be received from user accounts stored in the nutritional item recommendation server system 50, or the database device 58 thereof, or from plurality of user devices 10.

The method further comprises, receiving, in the nutritional item recommendation server system 50, nutritional item information of plurality of nutritional items, as nutritional item input, the nutritional item information comprising nutrition and ingredients information of plurality of nutritional items. The nutritional item input may be received from the network resources 102, 104, 106. Alternatively, the nutritional item input may be received from a nutritional item database in the nutritional item recommendation server system 50, or the database device 58 thereof.

In the nutritional item recommendation server system 50 it is provided the recommendation module 56 provided with the machine learning recommendation algorithm. The machine learning recommendation algorithm is then trained by applying, in the nutritional item recommendation server system 50, user input, meaning the personal information of plurality of users, and the nutritional item input, meaning the nutritional item information of plurality of nutritional items, as training data to the machine learning recommendation algorithm in the recommendation module 56 and generating, in the nutritional item recommendation server system 50 and in the recommendation module 56, a trained machine learning recommendation algorithm. Accordingly, the nutritional item recommendation system 1 and the nutritional item recommendation server system 50 is trained.

Once the nutritional item recommendation system 1 and the nutritional item recommendation server system 50, or the nutritional item recommendation algorithm is trained, the method comprises generating, by the trained machine learning recommendation algorithm of the nutritional item recommendation server system 50 or the recommendation module 56, a nutritional item recommendation output as a response to a recommendation request from a user based on the personal information of the user and the nutritional item information. The nutritional item recommendation request 20 may be received from the user device 10, optionally via the user interface 12 of the nutritional item recommendation application 14, via the communications network 100. The nutritional item recommendation server system 50, or the recommendation module 56 or the nutritional item recommendation algorithm, then generates nutritional item recommendation output for recommending a nutritional item for the user based on the personal information of the user and the nutritional item information. The nutritional item recommendation server system 50 then sends or transmits the nutritional item recommendation output 30, for example via the communications network 100, to the user device 10. The nutritional item recommendation output 30 may be displayed on the user interface 12 of the nutritional item recommendation application in the user device 10.

After receiving the nutritional item recommendation output, in the user device 10, the user may provide user feedback relating to the nutritional item recommendation output in the user interface 12. The user feedback information may be based on user action on the nutritional item recommendation output. The user feedback may comprise selecting, clicking or purchasing a nutritional item included on the nutritional item recommendation output in the user interface 12, or rating or commenting a nutritional item included on the nutritional item recommendation output in the user interface 12. The user feedback information is then sent or transmitted via the communications network 100 to the nutritional item recommendation server system 50 from the user device 10, or from nutritional item recommendation application 14 or the user interface 12.

Accordingly, the method comprises receiving, in the nutritional item recommendation server system 50, user feedback information from the user, or the user device 10, as a response to the nutritional item recommendation output. The method further comprises applying, in the nutritional item recommendation server system 50, the user feedback information as reinforcement training data to the recommendation module 56 or the machine learning recommendation algorithm of the recommendation module 56. Thus, the nutritional item recommendation system, or the recommendation module 56 or the machine learning recommendation algorithm is further trained with user feedback information for recommendations more suitable to a particular user.

It should be noted, that the user feedback information may be associated with a certain user or user account, and therefore the recommendation module 56 or the machine learning recommendation algorithm may take into account the user personal information together with the user feedback information for the training.

Accordingly, the machine learning recommendation algorithm may be a reinforcement machine learning algorithm or is arranged to implement reinforcement machine learning. Further, the machine learning recommendation algorithm is preferably an artificial neural network.

The method may further comprise receiving, in the nutritional item recommendation server system 50, user-item interaction information as user-item input, the user-item information comprising plurality of interactions associated between users and nutritional items. The user-item interaction input may be received from the network resources 102, 104, 106. Alternatively, the user-item input may be received from a user-item database in the nutritional item recommendation server system 50, or the database device 58 thereof, or from user accounts in the nutritional item recommendation server system 50, or the database device 58 thereof. The user-item information may comprise purchase history, search history, view history or click, rating, comment or the like information associated to nutritional items and to users. Each user-item interaction information is associated with a user. Further, the nutritional item recommendation server system 50 may store the user-item interaction information of users continuously to the database device 58 and/or in association of the user account of respective users.

The method also comprises applying, in the nutritional item recommendation server system 50, the user-item interaction information as training data to the machine learning recommendation algorithm and generating, in the nutritional item recommendation server system 50, the trained machine learning recommendation algorithm. Accordingly, the recommendation module 56 and the machine learning recommendation algorithm is trained with the user-item information.

In some embodiments, the method comprises receiving nutritional item selection information from plurality of users. The nutritional item selection information comprises one or more selected nutritional item and nutrition and ingredients information of the one or more selected nutritional items. The method further comprises applying the nutritional item selection information of plurality of users as reinforcement training data to the machine learning recommendation algorithm.

Alternatively, the method comprises maintaining nutritional item selection history of the plurality of users, and applying the nutritional item selection history of plurality of users as training data to the machine learning recommendation algorithm or as reinforcement training data to the machine learning recommendation algorithm.

Further alternatively, the method comprises maintaining nutritional item selection history of plurality of users and receiving nutritional item selection information from plurality of users. The nutritional item selection information comprising one or more selected nutritional item and nutrition and ingredients information of the one or more selected nutritional item. The method further comprises providing the nutritional item selection information from plurality of users to the nutritional item selection history of plurality of users, and applying the nutritional item selection information of plurality of users as training data to the machine learning recommendation algorithm or as reinforcement training data to the machine learning recommendation algorithm.

Thus, the machine learning recommendation algorithm may be trained with selections made by the users. This is valuable information together with the personal information and nutritional item information, and possible with healthiness information of the users.

The method may further comprise receiving, in the nutritional item recommendation server system 50, user electronic health records of the plurality of users as device input, the electronic health records comprising one or more measured physiological values of plurality of users. The device input may be received from one or more physiological measurement devices 110 via the communications network 100. Alternatively, the device input 110 may be received from the user device 10, or via the user interface 12 and the nutritional item recommendation application 14. In this case, the physiological measurement device 110 may be connected to the user device 10 and the user device 10 may receive the user electronic health records from the physiological measurement device 110. Further alternatively, the device input, meaning the user electronic health records, may be received from the one or more network resources 102, 104, 106 comprising the electronic health records. Further, the device input may be received from a nutritional item database in the nutritional item recommendation server system 50, or the database device 58 thereof. Each user electronic health record is associated with a user or user account.

The machine learning recommendation algorithm is then trained by applying, in the nutritional item recommendation server system 50, the device input, meaning the user electronic health records of plurality of users, as training data to the machine learning recommendation algorithm in the recommendation module 56 and generating, in the nutritional item recommendation server system 50 and in the recommendation module 56, a trained machine learning recommendation algorithm. Accordingly, the nutritional item recommendation system 1 and the nutritional item recommendation server system 50 is trained.

Once the nutritional item recommendation system 1 and the nutritional item recommendation server system 50, or the nutritional item recommendation algorithm is trained also with the user electronic health records, the method comprises generating, by the trained machine learning recommendation algorithm of the nutritional item recommendation server system 50 or the recommendation module 56, a nutritional item recommendation output as a response to a recommendation request from a user based on the personal information of the user, the nutritional item information and the user electronic health records.

The method may also comprise training the nutritional item recommendation system 1 and the recommendation module 56, and especially the machine learning recommendation algorithm with user healthiness information. The nutritional item recommendation server system 50 may comprise the healthiness module 60 for evaluating healthiness of plurality of users.

Accordingly, the method may comprise receiving, in the nutritional item recommendation server system 50, general medical information, the general medical information comprising general medical health records. The general medical information may be received from one or more of the network resources 102, 104, 106. Alternatively, the general medical information may be received from a medical information database in the nutritional item recommendation server system 50, or the database device 58 thereof. Thus, the general medical information may be received from an external resource or it may be stored in the nutritional item recommendation server system 50.

Then, the method may comprise providing, in the nutritional item recommendation server system 50, the healthiness module 60 comprising the machine learning grouping algorithm. The machine learning grouping algorithm is preferably a supervised learning algorithm or arranged to implement supervised learning. Further, the machine learning grouping algorithm may be an artificial neural network. Alternatively, the machine learning grouping algorithm may comprise the machine learning algorithm or machine learning healthiness algorithm, and a clustering algorithm. The clustering algorithm groups the users to a healthiness groups based on the output from the machine learning healthiness algorithm. Thus, the machine learning grouping algorithm may comprise two algorithms executed successively.

Then, the method comprises applying, in the nutritional item recommendation server system 50, the general medical information as training data to the machine learning grouping algorithm and providing, in the nutritional item recommendation server system 50, a trained machine learning grouping algorithm trained with the general medical information. The healthiness module 60 may thus comprise the trained machine learning grouping algorithm for evaluating healthiness of plurality of users based on user personal information, meaning the physiological information, and/or based on user electronic health records of plurality of users.

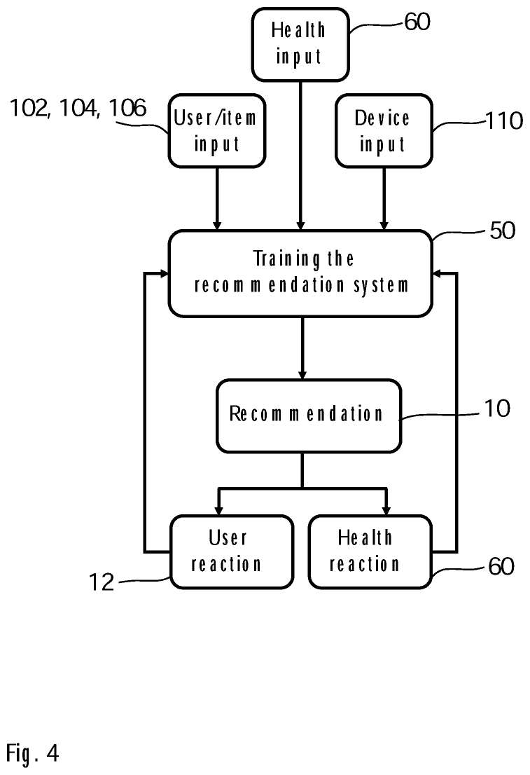

The healthiness module 60 or the machine learning grouping algorithm generates a user healthiness output when the user personal information and/or user electronic health records are applied as input data to the healthiness module 60 or the machine learning grouping algorithm. The user healthiness output comprises classification or grouping of a user to a healthiness group based on the user personal information and/or user electronic health record, by utilizing the machine learning grouping algorithm trained with the general medical information. The user healthiness output of plurality of users may be further applied as health input and training data to the recommendation module 56 or the machine learning recommendation algorithm, as shown in FIG. 4. The user healthiness output or the health input is associated with a user or a user account for training the nutritional item recommendation system, the recommendation module 56 and the machine learning recommendation algorithm.

In one embodiment of the present invention the method comprises receiving, in the nutritional item recommendation server system 50, personal information of the user and applying, in the nutritional item recommendation server system 50, the personal information of the user as grouping input data to the trained machine learning grouping algorithm. Then the method comprises generating, by the trained machine learning grouping algorithm of the nutritional item recommendation server system 50, a user healthiness output of the user based on the personal information of the user for grouping the user to a user healthiness group, and applying, in the nutritional item recommendation server system 50, the user healthiness output as health input, or as training data, to the machine learning recommendation algorithm and generating, in the nutritional item recommendation server system 50, the trained machine learning recommendation algorithm.

In an alternative embodiment of the present invention, the method may comprise receiving, in the nutritional item recommendation server system 50, personal information of the user and the electronic health records of the user, and applying, in the nutritional item recommendation server system 50, the personal information of the user and the electronic health records of the user as grouping input data to the trained machine learning grouping algorithm. Then the method comprises generating, by the trained machine learning grouping algorithm of the nutritional item recommendation server system 50, a user healthiness output of the user based on the personal information of the user and the electronic health records of the user for grouping the user to a user healthiness group, and applying, in the nutritional item recommendation server system 50, the user healthiness output as health input, or training data, to the machine learning recommendation algorithm and generating, in the nutritional item recommendation server system 50 the trained machine learning recommendation algorithm.

In a further alternative embodiment of the present invention, the method may comprise receiving, in the nutritional item recommendation server system 50, the electronic health records of the user, and applying, in the nutritional item recommendation server system 50, the electronic health records of the user as grouping input data to the trained machine learning grouping algorithm. Then the method may comprise generating, by the trained machine learning grouping algorithm of the nutritional item recommendation server system 50, a user healthiness output of the user based on the electronic health records of the user for grouping the user to a user healthiness group, and applying, in the nutritional item recommendation server system 50, the user healthiness output as training data to the machine learning recommendation algorithm and generating, in the nutritional item recommendation server system 50, the trained machine learning recommendation algorithm.

Accordingly, the user healthiness output from the healthiness module 60 may be based on the user personal information and/or the user electronic health records. Thus, the healthiness module 60 or the machine learning grouping algorithm trained with the general medical information classifies user to a healthiness group based on the user personal information and/or the user electronic health records. The user healthiness group is the health input to the recommendation module 56 or the machine learning recommendation algorithm.

In the nutritional item recommendation server system 50, may be maintained a pre-evaluated user healthiness outputs of plurality of users. The pre-evaluated user healthiness outputs may be stored in the nutritional item recommendation server system 50 or the database device 58 thereof. Each pre-evaluated user healthiness output may be stored or associated with user accounts of a respective user. The pre-evaluated user healthiness output may be generated for example upon creating a user account or it may be created periodically for example once a month or one a year, or upon request by a user. The pre-evaluated user healthiness output is stored in the nutritional item recommendation server system 50, the user device 10, and/or in association with the user account.

The pre-evaluated user healthiness output may be utilized training the recommendation module 60 and the machine learning recommendation algorithm. When a user requests a nutritional item recommendation the healthiness of the recommendation output may be evaluated by comparing the pre-evaluated user healthiness output and the healthiness output generated upon the recommendation request. The health reaction determined by the comparing is then applied as reinforcement training data to the machine learning recommendation algorithm.

The method may thus comprise upon the recommendation request from the user, receiving, in the nutritional item recommendation server system 50, personal information of the user, and generating, by the trained machine learning grouping algorithm of the nutritional item recommendation server system 50 or the healthiness module 60, the user healthiness output of the user by applying the personal information of the user to the machine learning grouping algorithm for grouping the user to the user healthiness group. Then the method comprises comparing, in the nutritional item recommendation server system 50, the pre-evaluated user healthiness output of the user and the user healthiness output of the user and generating, by the nutritional item recommendation server system 50, health reaction, or health feedback information, based on the comparison of the pre-evaluated user healthiness output of the user and the user healthiness output of the user generated upon the recommendation request. The method may further comprise applying, in the nutritional item recommendation server system 50, the health reaction, or the health feedback information, as reinforcement training data to the recommendation module 50 and the machine learning recommendation algorithm.

In an alternative embodiment, both the personal information of the user and the user electronic health records are used for generating, by the trained machine learning grouping algorithm of the nutritional item recommendation server system 50, the user healthiness output. Further alternatively, only the user electronic health records may be used for generating, by the trained machine learning grouping algorithm of the nutritional item recommendation server system 50, the user healthiness output.

The health reaction provided by the comparing is associated with a user or user account, and thus the health reaction may be associated further with the user-item interaction information of a user, and thus for example purchase history of the user. Therefore, the effects of the nutritional item recommendations or recommendation outputs on user healthiness may be taken into account and utilized in the training of the recommendation module or the machine learning recommendation algorithm.

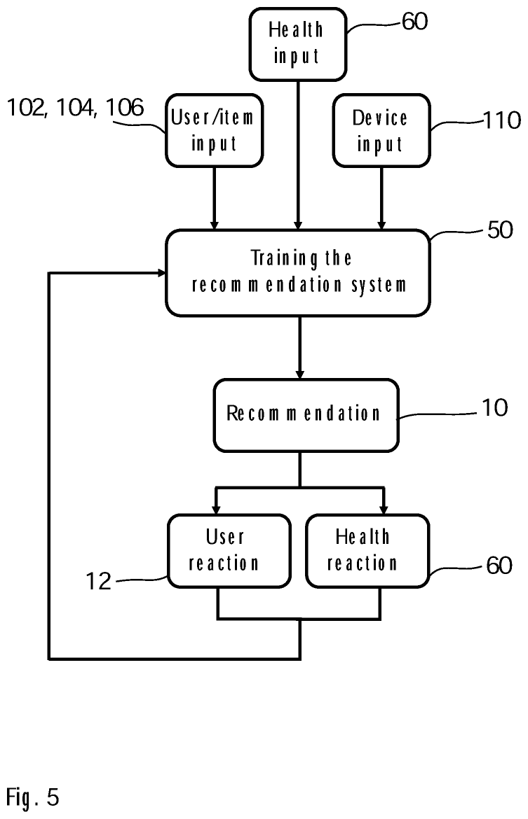

In the embodiment of FIG. 4, the user reaction or user feedback information, and the health reaction, or the health feedback information, are applied separately as reinforcement training data to the recommendation module 60 or the machine learning recommendation algorithm. It should be noted, that the user reaction and the health reaction may also be combined as combined feedback information or combined reaction. The combined feedback information may be applied in the nutritional item recommendation server system 50 as reinforcement training data to the recommendation module 50 and the machine learning recommendation algorithm, as shown in FIG. 5.

The trained nutritional item recommendation system 1 may then provide method for recommending nutritional items to a first user. Thus, the present invention provides a nutritional item recommendation method based on personal physical or physiological information of users and also based on the physical nutrition and ingredients data of nutritional items. The method utilizes the trained recommendation module and the trained machine learning recommendation algorithm, as disclosed above.

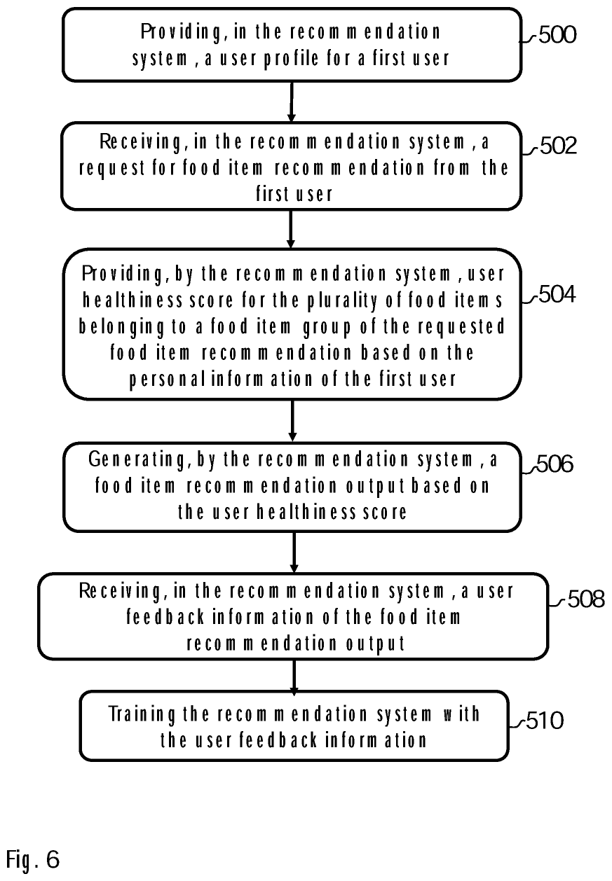

In the method there is maintained, in the nutritional item recommendation server system 50, plurality of user accounts, each user account comprising personal information of a user associated with the user account, the personal information comprising the personal physiological information, in step 500 of FIG. 6.

The method for recommending nutritional item to the first user comprises receiving in step 502, in the nutritional item recommendation server system 50, a nutritional item recommendation request from a first user associated with a first user account. The nutritional item recommendation request may be received from the user device 10 or via the user interface 12 of the nutritional item recommendation application 14. The nutritional item recommendation request and the personal information of the first user are applied, in the nutritional item recommendation server system 50, as recommendation input data to the trained machine learning recommendation algorithm in the recommendation module 56. Then the method comprises generating in step 506, by the trained machine learning recommendation algorithm of the recommendation server system 50 or the recommendation module 50, a nutritional item recommendation output as a response to the recommendation request from the first user based on the recommendation request and the personal information of the first user.

The method further comprises providing in step 504, by the recommendation server system 50, or the recommendation module or the machine learning recommendation algorithm, a user healthiness score for plurality of nutritional items belonging to a nutritional item group of the requested nutritional item recommendation based on the user personal information or the user profile or user account of the first user. The user healthiness score representing healthiness of a nutritional item to the first user. The nutritional item recommendation output is then generated based on the user healthiness scores of the plurality of nutritional items.

The method may further comprise receiving in step 508, in the recommendation server system 50, the user feedback information from the first user as a response to the nutritional item recommendation output, and applying in step 510, in the recommendation server system 50, the user feedback information as a reinforcement training data to the machine learning recommendation algorithm. The user feedback information may be received from the user device 10 or via the user interface 12 of the nutritional item recommendation application 14 as a response to user action, such a clicking, purchasing, viewing, rating or the like of one or more recommended nutritional items in the recommendation output.

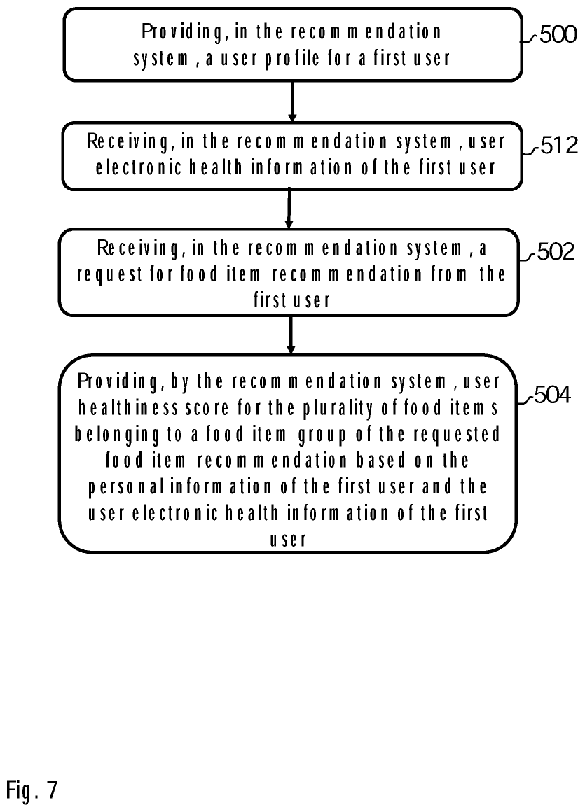

The method may also comprise receiving in step 512, in the nutritional item recommendation server system 50, user electronic health records of the first user, as shown in FIG. 7. The user electronic health records may be received in the nutritional item recommendation server system 50, as described above. The method may also comprise measuring, by the physiological measurement device 110, one or more physiological values of the first user, and then the measured physiological values of the first user are received as the user electronic health records of the first user in the nutritional item recommendation server system 50.

Then the user electronic health record of the first user are applied, in the nutritional item recommendation server system 50, as recommendation input data to the trained machine learning recommendation algorithm. The trained machine learning recommendation algorithm of the recommendation server system 50 or the recommendation module 56, generates the nutritional item recommendation output as the response to the recommendation request from the first user based on the recommendation request, the personal information of the first user and the electronic health record of the first user in step 506.

In this embodiment, method further comprises providing in step 512, by the recommendation server system 50, or the recommendation module 56 or the machine learning recommendation algorithm, the user healthiness score for plurality of nutritional items belonging to a nutritional item group of the requested nutritional item recommendation based on the user personal information or the user profile or user account of the first user and the user electronic health records of the first user.

It should be noted, that the machine learning recommendation algorithm of the recommendation module 56 utilized in the method may be trained by providing user electronic health records of plurality of users as training data to the machine learning recommendation algorithm of the nutritional item recommendation server system 50, the electronic health records comprising one or more measured physiological values of the plurality of users.

Furthermore and as described above, the machine learning recommendation algorithm may be trained by providing user-item interaction information as training data to the machine learning recommendation algorithm of the nutritional item recommendation server system 50, the user-item information comprising plurality of interactions associated between users and nutritional items. The nutritional item recommendation server system 50 may be configured to store user-item interaction information of the first user to the nutritional item recommendation server system 50 or the database device 58 thereof, or in association with the user account of the first user. The method may further comprise applying, in the recommendation server system 50, the user-item information as the reinforcement data or as training data to the machine learning recommendation algorithm.

The method may further comprise receiving nutritional item selection information from the first user. The nutritional item selection information comprises one or more selected nutritional item and nutrition and ingredients information of the one or more selected nutritional item.

Accordingly, the nutritional item selection information comprises past selections which the first user has made previously. Alternatively, the nutritional item selection information comprises the selection(s) made upon or after the nutritional item recommendation output and comprises the selection made from the one or more recommended nutritional items in the nutritional item category. The nutritional item selection information comprises nutrition and ingredients information of the selected nutritional item(s).

The method further comprises applying the nutritional item selection information of the first user as training data to the machine learning recommendation algorithm and generating.

In an alternative embodiment, the method comprises maintaining nutritional item selection history of the first user. The nutritional item selection history is associated to the first user account of the first user.

The nutritional item selection history comprises past selections of the first user stored to the nutritional item recommendation server system and associated to the first user or the first user account. This may be done receiving the nutritional item selection(s) made by the first user based on the nutritional item recommendation output and storing them to the nutritional item selection history data associated to the first user or the first user account. The nutritional item selection history comprises nutrition and ingredients information of the selected nutritional item(s).

The method further comprises applying the nutritional item selection history of the first user as training data to the machine learning recommendation algorithm.

In a further embodiment, the method comprises maintaining nutritional item selection history of the first user, receiving nutritional item selection information from the first user. The nutritional item selection information comprising one or more selected nutritional item and nutrition and ingredients information of the one or more selected nutritional item. The method further comprises providing the nutritional item selection information from the first user to the nutritional item selection history of the first user, and applying the nutritional item selection information of the first user as training data to the machine learning recommendation algorithm and generating.

The method of the present invention may further comprise maintaining nutritional item selection history of the first user, the nutritional item selection history being associated to the first user account of the first user, applying the nutritional item recommendation request, the personal information of the first user and the nutritional item selection history as input data to the trained machine learning recommendation algorithm, and generating a nutritional item recommendation output as a response to the recommendation request from the first user based on the recommendation request, the personal information of the first user and the nutritional item selection history, the nutritional item recommendation output comprising one or more nutritional items in the nutritional item group.

In an alternatively embodiment, the method comprises receiving nutritional item selection information from the first user. The nutritional item selection information comprises one or more selected nutritional items and nutrition and ingredients information of the one or more selected nutritional items. The method further comprises associating nutritional item selection information from the first user to the first user account, applying the nutritional item recommendation request, the personal information of the first user and the nutritional item selection information as input data to the trained machine learning recommendation algorithm, and generating by the trained machine learning recommendation algorithm a nutritional item recommendation output as a response to the recommendation request from the first user based on the recommendation request, the personal information of the first user and the nutritional item selection information. The nutritional item recommendation output comprises one or more nutritional items in the nutritional item group.

In a further embodiment, the method comprises maintaining nutritional item selection history of the first user. The nutritional item selection history is associated to the first user account of the first user. The method further comprises receiving nutritional item selection information from the first user. The nutritional item selection information comprises one or more selected nutritional item and nutrition and ingredients information of the one or more selected nutritional item. The method additionally comprises providing the nutritional item selection information from the first user to the nutritional item selection history of the first user, applying the nutritional item recommendation request, the personal information of the first user and the nutritional item selection history as input data to the trained machine learning recommendation algorithm, and generating, a nutritional item recommendation output as a response to the recommendation request from the first user based on the recommendation request, the personal information of the first user and the nutritional item selection history, the nutritional item recommendation output comprising one or more nutritional items in the nutritional item group.

The method may comprise evaluating healthiness of the first user upon recommendation request from first user, the evaluating healthiness of the first user being executable by the nutritional item recommendation server system 50 executing the trained machine learning grouping algorithm of the healthiness module 60. The trained machine learning grouping algorithm of the healthiness module 60 is trained by applying the general medical information as training data to the machine learning grouping algorithm of the healthiness module 60 or the nutritional item recommendation server system 50. The general medical information comprises general medical health records.

Thus, the present invention may comprise receiving, in the nutritional item recommendation server system 50, the personal information of the first user. The personal information of the first user may be received from the user device 10, or via the user interface 12 of the nutritional item recommendation application 14. Alternatively, the personal information of the first user may be stored in the nutritional item recommendation server system 50, or the database device 58 thereof, in association of the first user or the first user account of the first user.

Accordingly, the method may further comprise applying, in the nutritional item recommendation server system 50, the personal information of the first user as grouping input data to the trained machine learning grouping algorithm, and generating, by the trained machine learning grouping algorithm of the healthiness module 60 or the nutritional item recommendation server system 50, the user healthiness output of the first user based on the personal information of the first user. Accordingly, the healthiness output or classifying the first user to a healthiness group is carried out based on the personal information of the first user.

In an alternative embodiment, the method comprises receiving, in the nutritional item recommendation server system 50, electronic health records of the first user, in a manner as disclosed above. The method then further comprises applying, in the nutritional item recommendation server system 50, the electronic health records of the first user as grouping input data to the trained machine learning grouping algorithm, and generating, by the trained machine learning grouping algorithm of the healthiness module 60 or the nutritional item recommendation server system 50, the user healthiness output of the first user based on the electronic health records of the first user. Accordingly, the healthiness output or classifying the first user to a healthiness group is carried out based on the electronic health records of the first user.

In a further alternative embodiment, the method may comprise receiving, in the nutritional item recommendation server system 50, personal information of the first user and electronic health records of the first user, in a manner disclosed above. The method may further comprise applying, in the nutritional item recommendation server system 50, the personal information of the first user and the electronic health records of the first user as grouping input data to the trained machine learning grouping algorithm, and generating, by the trained machine learning grouping algorithm of the healthiness module 60 or the nutritional item recommendation server system 50, a user healthiness output of the first user based on the personal information of the first user and the electronic health records of the first user. Accordingly, the healthiness output or classifying the first user to a healthiness group is carried out based on the personal information of the first user and the electronic health records of the first user.

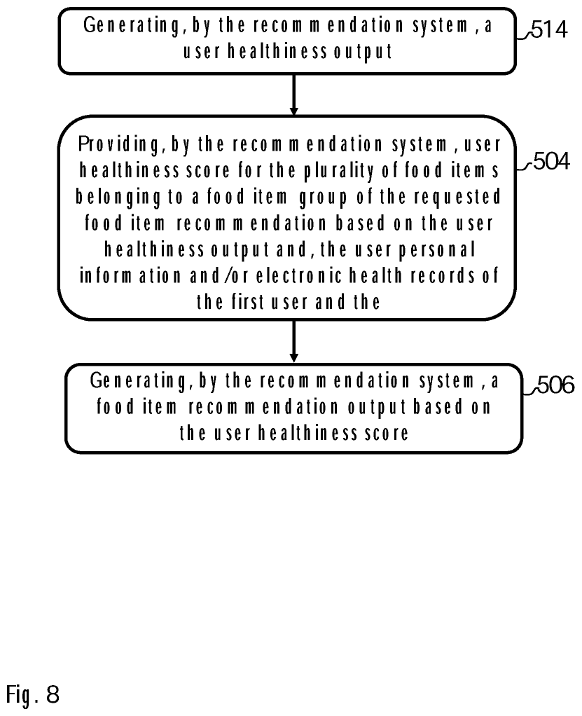

The healthiness module 60 or the machine learning grouping algorithm generates in step 514 the healthiness output of the first user, as shown in FIG. 8. The healthiness output comprises information on the healthiness of the first user such that the first user is classified or clustered to a healthiness group comprising users with similar healthiness characteristics.

The method may further comprise applying, in the nutritional item recommendation server system 50, the user healthiness output of the first user as recommendation input data to the trained machine learning recommendation algorithm. Therefore, the method comprises generating, by the trained machine learning recommendation algorithm of the healthiness module 60 of the nutritional item recommendation server system 50, the nutritional item recommendation output as the response to the recommendation request from the first user based on the recommendation request, the personal information of the first user and the user healthiness output of the first user, or based on the recommendation request, the personal information of the first user, the electronic health record of the first user and the user healthiness output of the first user, as shown in FIG. 8 in steps 504 and 506. Accordingly, the user healthiness score may be provided based on the recommendation request, the personal information of the first user and the user healthiness output of the first user, or based on the recommendation request, the personal information of the first user, the electronic health record of the first user and the user healthiness output of the first user. The user healthiness output of the first user is associated with the first user or with the first user account of the first user.

It should be noted, that the nutritional item recommendation output may be sent or transmitted or displayed in the user device 10, or in the user interface 12 of the nutritional item recommendation application 14. The nutritional item recommendation output may comprise recommended nutritional items provided based on the healthiness score, for example from highest healthiness score to lowest.

The invention has been described above with reference to the examples shown in the figures. However, the invention is in no way restricted to the above examples but may vary within the scope of the claims.

1.-24. (canceled)

25. A method for training a nutritional item recommendation system for recommending nutritional items for users, once trained the nutritional item recommendation system being arranged to automatically execute a machine learning recommendation algorithm, wherein the method comprises:
a) receiving, in a nutritional item recommendation server system, personal information of plurality of users, the personal information comprising physiological information of plurality of users;
b) receiving, in the nutritional item recommendation server system, nutritional item information of plurality of nutritional items, the nutritional item information comprising nutrition and ingredients information of plurality of nutritional items;
c) providing, in the nutritional item recommendation server system, machine learning recommendation algorithm;
d) applying, in the nutritional item recommendation server system, the personal information of plurality of users, the nutritional item information of plurality of nutritional items as training data to the machine learning recommendation algorithm and generating, in the nutritional item recommendation server system, a trained machine learning recommendation algorithm;
e) generating, by the trained machine learning recommendation algorithm of the nutritional item recommendation server system, a nutritional item recommendation output as a response to a recommendation request of the nutritional item from a user based on the personal information of the user and the nutritional item information, the nutritional item recommendation output comprising one or more recommended nutritional items;
f) receiving, in the nutritional item recommendation server system, user feedback information from the user as a response to the nutritional item recommendation output; and
g) applying, in the nutritional item recommendation server system, the user feedback information as reinforcement training data to the machine learning recommendation algorithm.

26. The method according to claim 25, wherein the method further comprises:
h) receiving, in the nutritional item recommendation server system, user-item interaction information, the user-item information comprising plurality of interactions associated between users and nutritional items; and
i) applying, in the nutritional item recommendation server system, the user-item interaction information as training data to the machine learning recommendation algorithm and generating, in the nutritional item recommendation server system, the trained machine learning recommendation algorithm.

27. The method according to claim 25, wherein the method further comprises:
receiving, in the nutritional item recommendation server system, nutritional item selection information from plurality of users in step f), the nutritional item selection information comprising one or more selected nutritional item and nutrition and ingredients information of the one or more selected nutritional items; and
applying, in the nutritional item recommendation server system, the nutritional item selection information of plurality of users as reinforcement training data to the machine learning recommendation algorithm in step g); or
maintaining, in the nutritional item recommendation server system, nutritional item selection history of the plurality of users; and
applying, in the nutritional item recommendation server system, the nutritional item selection history of plurality of users as training data to the machine learning recommendation algorithm in step d) or as reinforcement training data to the machine learning recommendation algorithm in step g); or
maintaining, in the nutritional item recommendation server system, nutritional item selection history of plurality of users;
receiving, in the nutritional item recommendation server system, nutritional item selection information from plurality of users in step f), the nutritional item selection information comprising one or more selected nutritional item and nutrition and ingredients information of the one or more selected nutritional item;
providing, in the nutritional item recommendation server system, the nutritional item selection information from plurality of users to the nutritional item selection history of plurality of users; and
applying, in the nutritional item recommendation server system, the nutritional item selection information of plurality of users as training data to the machine learning recommendation algorithm in step d) or as reinforcement training data to the machine learning recommendation algorithm in step g).

28. The method according to claim 25, wherein:
the step e) comprises generating, by the trained machine learning recommendation algorithm of the nutritional item recommendation server system, the nutritional item recommendation output as the response to the recommendation request of the nutritional item in a pre-determined nutritional item category from a user based on the personal information of the user and the nutritional item information, the recommendation request comprising a nutritional item category information of the pre-determined nutritional item category and the nutritional item recommendation output comprising one or more recommended nutritional items in the pre-determined nutritional item category; or
the step e) comprises:
generating, by the trained machine learning recommendation algorithm of the nutritional item recommendation server system, a nutritional item category based on the recommendation request of the nutritional item and the personal information of the user; and
generating, by the trained machine learning recommendation algorithm of the nutritional item recommendation server system, the nutritional item recommendation output as the response to the recommendation request of the nutritional item in the nutritional item category from a user based on the personal information of the user and the nutritional item information, the nutritional item recommendation output comprising one or more recommended nutritional items in the generated nutritional item category.

29. The method according to claim 25, wherein the step a) comprises:
receiving, in the nutritional item recommendation server system, user electronic health records of the plurality of users, the electronic health records comprising one or more measured physiological values of plurality of users; and
applying, in the nutritional item recommendation server system, the user electronic health records as training data to the machine learning recommendation algorithm and generating, in the nutritional item recommendation server system, the trained machine learning recommendation algorithm.

30. The method according to claim 25, wherein the method further comprises a step j):
receiving, in the nutritional item recommendation server system, general medical information, the general medical information comprising general medical health records;
providing, in the nutritional item recommendation server system, a machine learning grouping algorithm; and
applying, in the nutritional item recommendation server system, the general medical information as training data to the machine learning grouping algorithm and providing, in the nutritional item recommendation server system a trained machine learning grouping algorithm trained with the general medical information.

31. The method according to claim 30, wherein the method comprises a step k):
receiving, in the nutritional item recommendation server system, personal information of the user;
applying, in the nutritional item recommendation server system, the personal information of the user as grouping input data to the trained machine learning grouping algorithm;
generating, by the trained machine learning grouping algorithm of the nutritional item recommendation server system, a user healthiness output of the user based on the personal information of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm; or
receiving, in the nutritional item recommendation server system, personal information of the user and the electronic health records of the user;
applying, in the nutritional item recommendation server system, the personal information of the user and the electronic health records of the user as grouping input data to the trained machine learning grouping algorithm;
generating, by the trained machine learning grouping algorithm of the nutritional item recommendation server system, a user healthiness output of the user based on the personal information of the user and the electronic health records of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm; or
receiving, in the nutritional item recommendation server system, the electronic health records of the user;
applying, in the nutritional item recommendation server system, the electronic health records of the user as grouping input data to the trained machine learning grouping algorithm;
generating, by the trained machine learning grouping algorithm of the nutritional item recommendation server system, a user healthiness output of the user based on the electronic health records of the user for grouping the user to a user healthiness group; and
applying, in the nutritional item recommendation server system, the user healthiness output as training data to the machine learning recommendation algorithm and generating, in the nutritional item recommendation server system, the trained machine learning recommendation algorithm.

32. The method according to claim 30, wherein the method comprises step l):
maintaining, in the nutritional item recommendation server system, a pre-evaluated user healthiness output;
upon the recommendation request from the user, receiving, in the nutritional item recommendation server system, personal information of the user;
generating, by the trained machine learning grouping algorithm of the nutritional item recommendation server system, the user healthiness output of the user by applying the personal information of the user to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user; and
applying, in the nutritional item recommendation server system, the health feedback information as reinforcement training data to the machine learning recommendation algorithm; or
maintaining, in the nutritional item recommendation server system, a pre-evaluated user healthiness output;
upon the recommendation request from the user, receiving, in the nutritional item recommendation server system, personal information of the user and the user electronic health records;
generating, by the trained machine learning grouping algorithm of the nutritional item recommendation server system, the user healthiness output of the user by applying the personal information of the user and the user electronic health records to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user; and
applying, in the nutritional item recommendation server system, the health feedback information as reinforcement training data to the machine learning recommendation algorithm; or
maintaining, in the nutritional item recommendation server system, a pre-evaluated user healthiness output;
upon the recommendation request from the user, receiving, in the nutritional item recommendation server system, the user electronic health records;
generating, by the trained machine learning grouping algorithm of the nutritional item recommendation server system, the user healthiness output of the user by applying the user electronic health records to the machine learning grouping algorithm for grouping the user to the user healthiness group;
comparing, in the nutritional item recommendation server system, the pre-evaluated user healthiness output of the user and the user healthiness output of the user;
generating, by the nutritional item recommendation server system, health feedback information based on the comparison of the pre-evaluated user healthiness output of the user and the user healthiness output of the user; and
applying, in the nutritional item recommendation server system, the health feedback information as reinforcement training data to the machine learning recommendation algorithm.

33. A method for recommending a nutritional item for a first user, the method being executable by a nutritional item recommendation server system executing a machine learning recommendation algorithm, once trained, wherein the machine learning recommendation algorithm is trained by providing:
personal information of plurality of users as training data to the machine learning recommendation algorithm of the nutritional item recommendation server system, the personal information comprising physiological information of plurality of users; and
nutritional item information of plurality of nutritional items as training data to the machine learning recommendation algorithm of the nutritional item recommendation server system, the nutritional item information comprising nutrition and ingredients information of plurality of nutritional items,
the method comprising:
A) receiving, in the nutritional item recommendation server system, personal information of a first user, the personal information comprising the personal physiological information;
B) receiving, in the nutritional item recommendation server system, a nutritional item recommendation request of a nutritional item from the first user associated with a first user account;
C) applying, in the nutritional item recommendation server system, the nutritional item recommendation request and the personal information of the first user as input data to the trained machine learning recommendation algorithm; and
D) generating, by the trained machine learning recommendation algorithm of the recommendation server system, a nutritional item recommendation output as a response to the recommendation request from the first user based on the recommendation request and the personal information of the first user, the nutritional item recommendation output comprising one or more nutritional items.

34. The method according to claim 33, wherein:
the step A) comprises maintaining, in the nutritional item recommendation server system, plurality of user accounts, each user account comprising personal information of a user associated with the user account, the personal information comprising the personal physiological information; and
step B) comprises receiving, in the nutritional item recommendation server system, the nutritional item recommendation request of the nutritional item from the first user associated with the first user account.

35. The method according to claim 33, wherein:
the step D) comprises generating, by the trained machine learning recommendation algorithm of the nutritional item recommendation server system, the nutritional item recommendation output as the response to the recommendation request of the nutritional item in a pre-determined nutritional item category from the first user based on the personal information of the first user and the nutritional item information, the recommendation request comprising a nutritional item category information of the pre-determined nutritional item category and the nutritional item recommendation output comprising one or more recommended nutritional items in the pre-determined nutritional item category; or
the step D) comprises:
generating, by the trained machine learning recommendation algorithm of the nutritional item recommendation server system, a nutritional item category based on the recommendation request of the nutritional item and the personal information of the first user; and
generating, by the trained machine learning recommendation algorithm of the nutritional item recommendation server system, the nutritional item recommendation output as the response to the recommendation request of the nutritional item in the nutritional item category from the first user based on the personal information of the user and the nutritional item information, the nutritional item recommendation output comprising one or more recommended nutritional items in the generated nutritional item category.

36. The method according to claim 33, wherein the method further comprises step E) comprising:
receiving, in the recommendation server system, user feedback information from the first user as a response to the nutritional item recommendation output; and
applying, in the recommendation server system, the user feedback information as a reinforcement training data to the machine learning recommendation algorithm.

37. The method according to claim 33, wherein the method further comprises:
step F) of receiving, in the nutritional item recommendation server system, user electronic health records of the first user;
applying, in the nutritional item recommendation server system, the user electronic health record of the first user as input data to the trained machine learning recommendation algorithm in step C); and
generating, by the trained machine learning recommendation algorithm of the recommendation server system, the nutritional item recommendation output as the response to the recommendation request from the first user based on the recommendation request, the personal information of the first user and the electronic health record of the first user in step D); or
step G) of measuring, by a physiological measurement device (110), one or more physiological values of the first user;
step F) of receiving, in the nutritional item recommendation server system, the one or more measured physiological values of the first user as user electronic health records of the first user;
applying, in the nutritional item recommendation server system, the user electronic health records of the first user as input data to the trained machine learning recommendation algorithm in step C); and
generating, by the trained machine learning recommendation algorithm of the recommendation server system, the nutritional item recommendation output as response to the recommendation request from the first user based on the recommendation request, the personal information of the first user and the electronic health record of the first user in step D).

38. The method according to claim 33, wherein the machine learning recommendation algorithm is trained by providing:
user electronic health records of plurality of users as training data to the machine learning recommendation algorithm of the nutritional item recommendation server system, the electronic health records comprising one or more measured physiological values of the plurality of users.

39. The method according to claim 33, wherein:
the machine learning recommendation algorithm is trained by providing:
user-item interaction information as training data to the machine learning recommendation algorithm of the nutritional item recommendation server system, the user-item information comprising plurality of interactions associated between users and nutritional items; or
the machine learning recommendation algorithm is trained by providing:
user-item interaction information as training data to the machine learning recommendation algorithm of the nutritional item recommendation server system, the user-item information comprising plurality of interactions associated between users and nutritional items, and the method comprises;
receiving, in the nutritional item recommendation server system, user feedback information from the first user as the response to the nutritional item recommendation output;
applying, in the recommendation server system, the user feedback information as the reinforcement training data to the machine learning recommendation algorithm.

40. The method according to claim 33, wherein the method further comprises a step H) of evaluating healthiness of the first user upon recommendation request from first user, the evaluating healthiness of the first user being executable by the nutritional item recommendation server system executing a machine learning grouping algorithm, once trained, the machine learning grouping algorithm being trained by providing:
general medical information as training data to the machine learning grouping algorithm of the nutritional item recommendation server system, the general medical information comprising general medical health records, and the step H) comprising:
receiving, in the nutritional item recommendation server system, personal information of the first user;
applying, in the nutritional item recommendation server system, the personal information of the first user as grouping input data to the trained machine learning grouping algorithm;
generating, by the trained machine learning grouping algorithm of the nutritional item recommendation server system, a user healthiness output of the first user based on the personal information of the first user; or
receiving, in the nutritional item recommendation server system, electronic health records of the first user;
applying, in the nutritional item recommendation server system, the electronic health records of the first user as grouping input data to the trained machine learning grouping algorithm;
generating, by the trained machine learning grouping algorithm of the nutritional item recommendation server system, a user healthiness output of the first user based on the electronic health records of the first user; or
receiving, in the nutritional item recommendation server system, personal information of the first user and electronic health records of the first user;
applying, in the nutritional item recommendation server system, the personal information of the first user and the electronic health records of the first user as grouping input data to the trained machine learning grouping algorithm;
generating, by the trained machine learning grouping algorithm of the nutritional item recommendation server system, a user healthiness output of the first user based on the personal information of the first user and the electronic health records of the first user.

41. The method according to claim 40, wherein the method further comprise step I):
applying, in the nutritional item recommendation server system, the user healthiness output of the first user as recommendation input data to the trained machine learning recommendation algorithm; and
generating, by the trained machine learning recommendation algorithm of the nutritional item recommendation server system, the nutritional item recommendation output as the response to the recommendation request from the first user based on the recommendation request, the personal information of the first user and the user healthiness output of the first user, or based on the recommendation request, the personal information of the first user, the electronic health record of the first user and the user healthiness output of the first user.

42. The method according to claim 40, wherein the method further comprises step I):
maintaining, in the nutritional item recommendation server system, a pre-evaluated user healthiness output of the first user;
comparing, in the nutritional item recommendation server system, the pre-evaluated user healthiness output of the first user and the user healthiness output of the first user;
generating, by the nutritional item recommendation server system, health feedback information based on the comparison of the pre-evaluated user healthiness output of the first user and the user healthiness output of the first user;
applying, in the nutritional item recommendation server system, the health feedback information as reinforcement training data to the machine learning recommendation algorithm.

43. The method according to claim 33, wherein the machine learning recommendation algorithm is further trained by:
receiving, in the nutritional item recommendation server system, nutritional item selection information from the first user, the nutritional item selection information comprising one or more selected nutritional item and nutrition and ingredients information of the one or more selected nutritional item; and
applying, in the nutritional item recommendation server system, the nutritional item selection information of the first user as training data to the machine learning recommendation algorithm and generating; or
maintaining, in the nutritional item recommendation server system, nutritional item selection history of the first user, the nutritional item selection history being associated to the first user account of the first user; and
applying, in the nutritional item recommendation server system, the nutritional item selection history of the first user as training data to the machine learning recommendation algorithm; or
maintaining, in the nutritional item recommendation server system, nutritional item selection history of the first user, the nutritional item selection history being associated to the first user account of the first user;
receiving, in the nutritional item recommendation server system, nutritional item selection information from the first user, the nutritional item selection information comprising one or more selected nutritional item and nutrition and ingredients information of the one or more selected nutritional item;
providing, in the nutritional item recommendation server system, the nutritional item selection information from the first user to the nutritional item selection history of the first user; and
applying, in the nutritional item recommendation server system, the nutritional item selection information of the first user as training data to the machine learning recommendation algorithm and generating.

44. The method according to claim 33, wherein method comprises step J):
maintaining, in the nutritional item recommendation server system, nutritional item selection history of the first user, the nutritional item selection history being associated to the first user account of the first user;
in step C) applying, in the nutritional item recommendation server system, the nutritional item recommendation request, the personal information of the first user and the nutritional item selection history as input data to the trained machine learning recommendation algorithm; and
in step D) generating, by the trained machine learning recommendation algorithm of the recommendation server system, a nutritional item recommendation output as a response to the recommendation request from the first user based on the recommendation request, the personal information of the first user and the nutritional item selection history, the nutritional item recommendation output comprising one or more nutritional items in the nutritional item group; or
receiving, in the nutritional item recommendation server system, nutritional item selection information from the first user, the nutritional item selection information comprising one or more selected nutritional items and nutrition and ingredients information of the one or more selected nutritional items;
associating, in the nutritional item recommendation server system, nutritional item selection information from the first user to the first user account; and
in step C) applying, in the nutritional item recommendation server system, the nutritional item recommendation request, the personal information of the first user and the nutritional item selection information as input data to the trained machine learning recommendation algorithm; and
in step D) generating, by the trained machine learning recommendation algorithm of the recommendation server system, a nutritional item recommendation output as a response to the recommendation request from the first user based on the recommendation request, the personal information of the first user and the nutritional item selection information, the nutritional item recommendation output comprising one or more nutritional items in the nutritional item group; or
maintaining, in the nutritional item recommendation server system, nutritional item selection history of the first user, the nutritional item selection history being associated to the first user account of the first user;
receiving, in the nutritional item recommendation server system, nutritional item selection information from the first user, the nutritional item selection information comprising one or more selected nutritional item and nutrition and ingredients information of the one or more selected nutritional item;
providing, in the nutritional item recommendation server system, the nutritional item selection information from the first user to the nutritional item selection history of the first user;
in step C) applying, in the nutritional item recommendation server system, the nutritional item recommendation request, the personal information of the first user and the nutritional item selection history as input data to the trained machine learning recommendation algorithm; and
in step D) generating, by the trained machine learning recommendation algorithm of the recommendation server system, a nutritional item recommendation output as a response to the recommendation request from the first user based on the recommendation request, the personal information of the first user and the nutritional item selection history, the nutritional item recommendation output comprising one or more nutritional items in the nutritional item group.
